(19) United States
(12) Patent Application Publication
(10) Pub. No.: US 2025/0235482 A1
(43) Pub. Date: Jul. 24, 2025

(54) PRODUCTION AND THERAPEUTIC USE OF OFF-THE-SHELF DOUBLE NEGATIVE T CELLS

(71) Applicant: University Health Network, Toronto (CA)

(72) Inventors: Li Zhang, Toronto (CA); Jong Bok Lee, Toronto (CA); Hyeonjeong Kang, North York (CA)

(21) Appl. No.: 19/172,018

(22) Filed: Apr. 7, 2025

Related U.S. Application Data
(63) Continuation of application No. 17/415,957, filed on Jun. 18, 2021.
(60) Provisional application No. 62/782,005, filed on Dec. 19, 2018.

(51) Int. Cl. A61K 35/28; A01N 1/125; A61K 39/395; A61K 40/11; A61K 40/46; A61K 40/50; C07K 16/28; C12N 5/0783

(52) U.S. Cl. CPC A61K 35/28; A01N 1/125; A61K 39/3955; A61K 40/11; A61K 40/46; C07K 16/2809; C12N 5/0636; A61K 40/50; A61K 2239/31; A61K 2239/38; A61K 2239/48; C12N 2500/92; C12N 2501/2302; C12N 2501/515

(57) ABSTRACT

Described are methods for the production and use of cryopreservable double negative T cells (DNTs) for the treatment of cancer as an off-the-shelf cellular therapy. A sample population of DNTs is expanded using DNTs from one or more donors. The expanded population of DNTs from different donors does not exhibit alloreactivity against allogenic cells in the expanded population. The expanded populations of DNTs can be long-term stored as cryopreserved products.

### CROSS-REFERENCE TO RELATED APPLICATIONS

The present application is a continuation of U.S. Ser. No. 17/415,957 filed on Jun. 18, 2021, that is a national phase entry of PCT/CA2019/051866 filed Dec. 19, 2019 which claims priority to U.S. provisional patent application No. 62/782,005 filed Dec. 19, 2018, the entire contents of which are hereby Incorporated by reference.

### FIELD OF THE INVENTION

The present invention relates to double negative T cells (DNTs) and more specifically to the preparation of cryopreservable DNTs and the use of DNTs as an off-the-shelf adoptive cellular therapy for the treatment of cancer.

### BACKGROUND OF THE INVENTION

The effectiveness of adoptive cellular therapy (ACT) using T cells to treat different hematological and solid malignancies has been demonstrated in multiple clinical studies.^{1,2} Advances in technologies such as genetically modifying immune cells to express a chimeric antigen receptor (CAR) or a transgenic-T cell receptor and use of artificial antigen presenting cells have been implemented to improve the therapeutic potency of ACTs.^{3,4} Recently, CD19-CAR T cell therapy achieved effective clinical responses in patients with B cell malignancies¹ and has been FDA approved for clinical use for these diseases⁵. However, with increasing numbers of patients needed to be treated with ACT, limitations of current forms of ACT have become apparent, including sophisticated expansion methods resulting in uncertainty of producing therapeutically relevant numbers of T cells, time required for cell expansions, requirement of clinically-approved facilities for cell expansion, inconsistency of manufactured cellular products, and high production costs.⁶

Allogeneic hematopoietic stem cell transplantation (allo-HSCT) is the standardized second line of treatment with a long-term curative potential for patients with hematopoietic malignancies of multiple types.⁷ Therapeutic benefit of allo-HSCT comes from donor-derived immune cell-mediated graft-versus-leukemia (GvL) effect targeting leukemic blasts that are resistant to conventional induction chemotherapies.⁷ Improved survival in patients receiving allo-HSCT demonstrates the potency of immune cell mediated GvL effect, but the effect is incomplete. To boost the GvL effect, patients can be treated with donor lymphocyte infusion (DLI), where mature lymphocytes from the periphery of the HSC donor is given to the transplant-recipient as a prophylactic or therapeutic regimen to prevent or to treat the disease relapse post-transplant.⁸ Yet, recurrent disease remains the leading cause of mortality and is seen in 30-40% of allo-HSCT patients.⁹

Further, allo-HSCT Is associated with high treatment-associated toxicities. Of those, graft-versus-host disease (GvHD) is the leading cause of non-relapse mortality (NRM) in patients receiving allo-HSCT.^{10,11} GvHD occurs through donor-derived immune cells recognizing the normal allogenelc tissues of the recipients as foreign and attacking them. Acute GVHD is seen in 30-50% of treated patients with 14% suffering from more severe grade III or grade IV and chronic GvHD is manifested in 30-70% of allo-HSCT recipients.^{10,11} GvHD significantly compromises patients' quality of life and increase their morbidity and mortality. Unfortunately, currently available immunosuppressant targets donor-derived T cells without distinguishing those inducing GvL effects and GvHD. Therefore, the side-effects of current forms of immunosuppressants include increased risk of disease relapse and infections. Therefore, treatments that can induce GYL without GvHD or control GvHD while maintaining GvL when used in adjuvant with allo-HSCT are the ‘Holy-Grail’ for allo-HSCT patients.

Off-the-shelf ACT focuses on generating large batches of cells from allogeneic donors and using them to treat a large array of patients.¹² As this approach is not patient-specific, cellular products can be pre-manufactured to save time.^{6,12} Mass production also increases product consistency, availability, and reliability at a lower cost. However, an effective clinically-applicable off-the-shelf allogenic T cell therapy should meet the following criteria: 1) expandable to a therapeutically relevant number under clinically-compliant condition; 2) do not cause graft vs. host disease (GvHD); 3) are able to target an array of cancers in a donor-unrestricted manner; 4) are not rejected by recipient's immune system to enable sufficient persistence; 5) can be stored under GMP conditions without hampering their function. To best of our knowledge, there has not been a report to show an adoptive cellular therapy that has capacity of long-term storage and can avoid host-versus-graft (HvG) rejection while fulfilling other requirements of off-the-shelf therapy without any genetic modification.

Double negative T cells (DNT) are mature T cells that comprises 3˜5% of peripheral T cells and is defined by expression of CD3 in the absence of CD4 and CD8.¹³⁻¹⁵ Recently, healthy donor (HD) derived-allogeneic DNTs were demonstrated to target acute myeloid leukemia (AML) in vitro and in patient-derived xenograft models and to have synergistic anti-cancer activities with conventional chemotherapies.¹³⁻¹⁵

### SUMMARY OF THE INVENTION

In one aspect, the inventors have developed methods to expand DNTs to therapeutic levels under GMP conditions that can be cryopreserved for long-term storage and characterized their surface molecule expression pattern using flow cytometry-based high throughput screening. The off-the-shelf potential of clinical-grade DNTs was investigated by assessing cytotoxicity induced by DNTs of various donor origin against multiple cancer types and their off-tumor toxicity in vitro and in xenograft models and determining the effect of cryopreservation under GMP conditions on cell viability and function. Further, the susceptibility of DNTs to conventional allogeneic T cells in vitro and in vivo was determined.

In one aspect, the inventors investigated the application of off-the-shelf DNTs as a monotherapy or as an adjuvant to allogeneic hematopoletic stem cell transplant (allo-HSCT) to treat cancer. DNTs-infused with peripheral mononuclear cells (PBMC) showed superior anti-leukemic activity than DNT-monotherapy and showed reduced off-tumor toxicities than PBMC-monotherapy in xenograft models.

Remarkably, as shown in Example 1 clinical-grade DNTs expanded 1558±795.5 fold in 17 days with >90% purity. Expanded DNTs showed potent in vitro cytotoxic activity against various cancer types in a donor-unrestricted manner, where DNTs from a single donor targeted multiple leukemia targets and DNTs from various donors show similar degree of anti-leukemia activity against same targets. DNTs enhanced the survival of mice infused with a lethal dose of Epstein-Barr virus transformed lymphoblastoid cell line (EBV-LCL) and significantly reduced leukemia engraftment in human leukemia-xenograft models. The inventors established a protocol to expand clinical-grade cryopreserveable DNTs and a protocol to optimally cryopreserve them using GMP-compliant reagents that maintained viability and anti-cancer functions for at least 600 days. Importantly, live allogeneic DNTs did not induce cytotoxicity of allo-reactive CD8⁺ T cells in vitro, and co-infusion of live DNTs with PBMC from a different donor into mice resulted in co-engraftment of DNTs and PBMC-derived allogeneic conventional T cells in the absence of cytotoxicity towards DNTs, suggesting the lack of host-versus-graft reaction. The methods described herein are therefore useful for generating therapeutic numbers of cryopreservable clinical-grade DNTs that fulfill the requirements of an off-the-shelf adoptive cell therapy.

As shown in Example 2, clinical-grade DNTs can be expanded from multiple different (allogenic) donors in the same culture without developing alloreactivity to each other during expansion. Characterization of the DNTs expanded ex vivo from pooled donor samples showed that the expanded allogenic cells maintained cytotoxicity against cancer cells without inducing cytotoxicity against normal cells or causing graft versus host disease (GvHD). Accordingly, in one embodiment there is provided a method of expanding a population of double negative T cells (DNTs) ex vivo, the method comprising:

a) providing a sample population of DNTs, wherein the sample population of DNTs comprises DNTs from one or more donors; and

b) culturing the sample population of DNTs in a culture media to produce an expanded population of DNTs, optionally a clinical-grade population of DNTs.

In one embodiment, the sample population of DNTs comprises DNTs from two or more donors. In one embodiment, the sample population of DNTs comprises DNTs from peripheral blood, leukapheresis, Leukopak, bone marrow and/or cord blood samples

In one embodiment, the DNTs from different donors are not alloreactive against one another in the expanded population of DNTs. In one embodiment, DNTs from different donors in the sample population are not alloreactive against each other. In one embodiment, the culture media is animal serum-free media. In one embodiment, the culture media further comprises human blood-derived components, optionally human plasma, serum, or HSA. The human-blood-derived components may be autologous to the sample population of DNTs or allogenic. Optionally, the human-blood-derived components comprise plasma from one or more donors. In one embodiment, the concentration of human-blood-derived components in the culture media is about 1-20%. In on embodiment, the concentration of plasma in the culture media is 2-15%. In one embodiment, the sample population of DNTs comprises DNTs from peripheral blood. In one embodiment the expanded population of DNTs yields at least 0.1, 0.2, 0.5, 0.8 or 1.0×10⁸ DNTs per milliliter of peripheral blood.

In one embodiment, the expanded population of DNTs comprises or consists of at least 50%, 60%, 70%, 80%, 85% or 90% DNTs. I none embodiment, the method comprises splitting the cells to maintain a cell population above 0.1 million per ml of the culture media and below 4 million per ml of the culture media.

As shown Example 3, further investigations into the long-term cryopreservation of DNTs identified cryopreservation methods that preserved the viability and cytotoxic activity of the cells for at least 600 days.

In one embodiment, there is provided a method of producing a population of double negative T cells (DNTs) for therapeutic applications. In one embodiment, the method comprises:

providing a sample population of DNTs, wherein the sample population of DNTs comprises DNTs from one or more donors;

culturing the sample population of DNTs in a culture media to produce an expanded population of DNTs, optionally Wherein the culture media is GMP-compliant;

re-suspending the expanded population of DNTs in a storage medium, and optionally

adding DMSO to the storage medium to a final concentration of between about 3% and about 15% DMSO, optionally between about 5% and 10% DMSO.

In one embodiment, the method comprises adding DMSO to the storage medium to a final concentration of between about 3% and about 15% DMSO, optionally between about 5% and 10% DMSO

Also provided is a method for cryopreserving DNTs. In one embodiment, the method comprises:

a) re-suspending a population of expanded DNTs in a storage medium;

b) adding DMSO to the storage medium to a final concentration of between about 3% and about 15% DMSO; and

c) cryopreserving the population of DNTs in the storage medium at a temperature less than −70° C.

In one embodiment, the population of DNTs has been expanded ex vivo, optionally according to a method for expanding DNTs as described herein, prior to re-suspending the population of DNTs in the storage medium.

In one embodiment, the final concentration of DMSO in the storage medium is from about 3% to about 15%, optionally from about 5% to 10%. In one embodiment, DMSO is added to the storage medium. In one embodiment, the DNTs are at a final concentration in the storage medium of between about 2.5×10⁷ and about 2.5×10⁸ cells/ml, optionally between about 5 and 10×10⁷ cells/ml.

Also provided is a population of DNTs produced, expanded and/or cryopreserved according to a method described herein. In one embodiment, the population is from a single expansion of DNTs from one or more donors and is for use or administration in one or more subjects for the treatment of cancer. In one embodiment, the population of DNTs is from a single expansion of DNTs from one or more donors and is for use or administration in one or more treatments for one subject with cancer. In one embodiment, the population of DNTs comprises DNTs from two or more donors and is for use or administration for the treatment of cancer.

In one embodiment, the population of DNTs express CD3 and do not express CD4 or CD8 prior to expansion, and/or express CD3 and do not express CD4 or CD8 at least 5 days, 10 days, 14 days, 17 days, or 20 days post expansion.

In one embodiment, the population of DNTs are CD11a+, CD18+, CD10−, and/or TCR Vα24−Jα18−. In one embodiment, the population of DNTs are

DNTs are CD49d+, CD45+, CD58+ CD147+ CD98+ CD43+ CD66b− CD35− CD36− and/or CD103−.

In another aspect, there is provided a method of treating cancer in a subject in need thereof, the method comprises administering to the subject an effective amount of a population of DNTs as described herein, optionally in combination with allo-HSCs and/or PBMCs. In one embodiment, the population of DNTs comprises allogenic DNTs from one or more donors, optionally two or more donors. Also provided is the use of a population of DNTs for treating cancer as a monotherapy or in combination with allo-HSCs and/or PBMCs, wherein the population of DNTs comprises allogenic DNTs from one or more donors, optionally two or more donors. In one embodiment, the methods and uses described herein comprise the administration or use of DNTs as a monotherapy. In one embodiment, the methods and uses described herein comprise the administration or use of DNTs and allogenic HSCs and/or PBMCs at the same time. In another embodiment, the methods and uses described herein comprises the administration or use of DNTs and allogenic HSCs and/or PBMCs at different times. Remarkably, as shown in Example 3 and FIG. 18B, AML cells were not detectable in bone marrows of mice treated with PBMC followed by DNTs in an NSG xenograft mouse model of AML.

Also provided is a composition or kit comprising DNTs and HSCs. Also provided is a composition or kit comprising DNTs and PBMCs. In one embodiment, the PBMCs are lymphocytes such as conventional CD4+ CD8+ T cells. In one embodiment, the DNTs described herein are for use in combination with donor lymphocyte infusion for the treatment of cancer in a subject in need thereof. In one embodiment, the kit comprises DNTs and HSCs and/or PBMCs in in different containers.

In one embodiment, the DNTs have been expanded ex vivo, optionally wherein the allogenic DNTs have been expanded according to a method described herein.

In one embodiment, the DNTs from different donors are not alloreactive against each other in the population of DNTs. In one embodiment, the population of DNTs is resistant to allogenic immune cell-mediated rejection in the subject in vivo. In one embodiment, the population of DNTs persists in vivo in the subject for at least 10 days, optionally for at least 2 weeks, at least 3 weeks, or at least 4 weeks.

Also provided are kits for expanding and/or cryopreserving a population of DNTs as described herein.

### BRIEF DESCRIPTION OF THE DRAWINGS

Embodiments of the invention will now be described in relation to the drawings in which:

FIG. 1. Clinical-grade DNTs expanded under GMP conditions. (A and B) Number of DNTs derived from each ml blood (A) and fold expansion (B) after 17 days culture are shown. Each symbol represents the result from one of 13 DNT cultures derived from 11 different donors C) DNTs expanded as described herein were stained with immune cell subset markers: CD3, CD4, and CD8 to check the purity of cells. Result shown is a representative of DNTs expanded from 13 expansions. (D-I) Results of flow-cytometry based surface molecule high-throughput screening on expanded DNTs from three donors are shown. Histograms show representative results for T-cell associated markers, CD2, CD3, and CD5, and B cell associated markers, CD19 and CD20, to confirm the validity of the screening method (D). Graphs show expression of T cell differentiation markers (E), chemokine receptors (F) cytotoxic (G), co-stimulatory (H), and co-inhibitory (I) molecules on expanded DNTs from three donors. Each symbol represents DNTs from one donor. Numbers shown are % of cells that expressed corresponding molecules on DNTs. Horizontal bars represent the mean±SEM. (J) Addition of TIM-3 antibody reduced the level of killing mediated by DNTs against AML3/OCI. (K) Addition of anti-CD3 antibody increased the killing mediated by DNTs against AML3/OCL

FIG. 2. DNTs induce cytotoxic activity against various cancer targets without off-tumor toxicity. A) Cytotoxicity of DNTs expanded from HDs. against cell lines derived from various cancer types: myeloma (82), T cell leukemia (Jurkat), Burkitt's lymphoma (Daudi), AML (OCI/AML3), EBV-LCL, large cell lung cancer (H460), and adenocarcinoma (A549) using the in vitro flow cytometry-based killing assay as described herein. Experiments were done in triplicates and results shown are representative of more than 3 independent experiments for each target. B) In vitro killing assay performed using DNTs expanded from two HDs (HD1 and HD2) against two leukemia cell lines, OCI/AML3and MV4-11, and a primary AML patient sample show that DNTs from a single donor kill multiple cancer targets. Experiments were done in triplicates. Result shown is representative of three separate experiments. C) In vitro killing assay were done using DNTs expanded from six HD s against the same cancer target, OCI/AML3, showing donor unrestricted activity of DNTs. Experiments were done in triplicates. The result shown is representative of three similar independent experiments. (D and E) NSG mice engrafted with EBV-LCL (D) or MV4-11 (E) were treated with three infusions of DNTs or PBS. D) Survival of EBV-LCL infused mice treated with DNT (n=6) or PBS (n=6) was monitored. Result shown is representative of three separate experiments done with DNT s from different donors. E) AML engraftment level in bone marrow was determined. Result shown is representative of four separate experiments. Each dot represents result from one mouse and horizontal bars represent the mean values±SEM of each group. F) In vitro killing assay conducted against primary AML patient sample containing leukemic blasts and normal cells. Left flow panels show the gating strategy used to distinguish leukemic from normal cells. Histogram shows the absence of off-tumor toxicity mediate d by DNTs while inducing potent cytotoxicity towards cancerous cells. Experiments were done in triplicates. Result shown is representative of four independent experiments done with different patient samples. (G and H) NSG mice inoculated with AML cell line MV4-11 were treated with PBS, human DNTs or PBMCs. 28 days post injection of AML, mice were euthanized and the liver and lung tissues were formalin fixed and stained with hematoxylin and eosin (H&E). G) Representative H&E stained slides of liver (400× magnification) and lung (200× magnification) from each group are shown. White arrows indicate bile ducts, grey arrows indicate bronchioles, and black arrows indicate the vessels. PV—portal vein; alv—alveoli. H) Tissue damage of H&E stained lung (left) and liver (right) slides were blindly scored by a pathologist. Each dot represent one mouse and horizontal bar represents the mean±SEM.

Data shown are representative of four separate experiments. **, p<0.01; ***, p<0.001 ****, p<0.0001, using unpaired, two-tailed Student's t test.

FIG. 3. Allogeneic DNTs can be cryopreserved under clinically-compliant conditions while maintaining their function. (A and B) Ex vivo expanded DNTs were cryopreserved using an animal serum free reagent as described herein. The % viability (A) and in vitro cytotoxicity (B) of DNTs after freezing and thawing (FT) were compared to DNTs from the same expansion culture without FT C) Cryopreserved DNTs were used to treat NSG mice that were pre-infused with MV4-11 and the level of engraftment was determined in the bone marrow, as described in FIG. 2E. D) Shelf-life of cryopreserved DNTs were determined by thawing DNTs frozen for different durations (617, 534, 276, 129, and 8 days) and checking their viability (left) and cytotoxicity (right) against a known DNT-susceptible cancer target (OCI/AML3). Data shown is representative of three similar experiments.

FIG. 4. DNTs can persist in vitro and in vivo in the presence of allogeneic CD4⁺ and CD8⁺ T cells. (A-C) CFSE-labeled ex vivo expanded DNTs were intravenously Injected into sublethally irradiated NSG mice (n=12). On days indicated, cells from blood, spleen, bone marrow (BM), liver, and lung were stained with anti-human CD45 and CD3 antibodies, and DNTs were detected by flow cytometry (A, n=3 per day). CFSE median fluorescence intensity (MFI) of DNTs on days 0, 2, 7, 10, and 14 post injection was measured by flow cytometry. Histogram (B) and relative reduction of CFSE MFI with respect to day 0 CFSE MFI (C) are shown. The results shown the results obtained from 3 mice per time point and are representative of two separate experiments using DNTs from two different HDs. (D-G) Mixed lymphocyte reaction (MLR) was conducted using HD1 PBMC and HD2 expanded DNTs to determine the immunogenicity of expanded DNTs to allogeneic T cells. D) Schematic diagram shows the MLR conducted. E) CFSE-labeled or unlabeled HD1 PBMC were co-cultured with live or irradiated expanded HD1 or HD2 DNT for 4-6 days. At the end of the MLR, % increase in proliferating cells compared to the unstimulated control was determined as described herein. Left histogram shows the representative CFSE dilution, gated on CD8⁺ T cells. Experiments were done in triplicates, and the bar graph on the right shows the average of the triplicates. The results are representative of 2 separate experiments using different HDs for autologous and 5 separate experiments using 4 different HDs pairs for allogeneic DNTs. F) The level of cytotoxicity against DNTs by CD8⁺ T cells stimulated with live or irradiated autologous or allogeneic DNTs was determined. HD1 CD8⁺ T cells isolated post MLR were co-cultured with autologous (empty) or allogeneic (filled) DNTs at varying effector to target ratios. Results shown are representative of 5 independent experiments using 4 pairs of donors for allogeneic DNTs and 2 independent experiments with 2 pairs of donors for autologous DNTs. G) Sublethally irradiated mice were infused with HLA-A2⁺ PBMC and HLA-A2⁻ DNTs (n=5). 28 days post infusion, mice were sacrificed and cells from lungs were stained with human anti-CD45, anti-HLA-A2, anti-CD3, anti-CD4, and anti-CD8 antibodies and DAPI to determine the engraftment of human T cell subsets. Numbers represent the % of cells in the corresponding gates. Bar graphs show the frequency of HLA-A2⁺ CD4⁺/CD8⁺ T cells and HLA-A2⁻ DNTs in the lungs. Each dot represents a single mouse. Result shown is representative of two separate experiments.

FIG. 5. Characterization of healthy donor (HD) DNT expansion using GMP-grade reagents. DNTs were expanded ex vivo with GMP-grade reagents including two types of animal-serum free media (AIM V and GT-T551). (A and B) Expansion profile (A) and purity (B) of DNTs from the same donor using two different culture media. C) Cytotoxicity of DNTs expanded using two types of media against OCI/AML3 and MV4-11. The results are representative of 3 experiments using 3 HDs. *, p<0.01.

FIG. 6. Mixing of DNTs from two different donors retains anti-leukemic function without alloreactivity against each other. A) In vitro flow cytometry based killing assay conducted against AML cell line using HLA-A2⁻ DNTs, HLA-A2⁺ DNTs, and the two donor DNTs mixed at 1:1 ratio. B) % dead DNTs from each donor with or without mixing was determined by flow cytometry after 2-hour co-incubation. The results are representative of two separate experiments using two different sets of HD DNTs.

FIG. 7. Co-engrafted allogeneic CD8⁺ T cells are not cytotoxic against DNTs. Sublethally irradiated mice were infused with HLA-A2⁺ PBMC and HLA-A2⁻ DNTs. Four weeks post PBMC infusion, mice were sacrificed and cells from spleens were pooled and HLA-A2⁺ CD8⁺ T cells were isolated, Isolated CD8⁺ T cells were used as effector cells against the HLA-A2⁻ DNTs originally used for xenograft experiment in an in vitro killing assay at 4:1 CDB: DNT for 14 hours. Flow plots show the viability of HLA-A2⁻ DNTs with or without coculture with HLA-A2⁺ CD8⁺ T cells. Result shown is representative of two separate experiments.

FIG. 8. Off-the-shelf potential of allogeneic DNTs. A) DNTs expanded from different HDs show similar levels of cytotoxicity against the same AML blasts. Killing assays were done by using DNTs expanded from 6 HDs as effectors against leukemia cells, B) Killing assay performed using DNTs expanded from 2 different HDs against primary and immortalized AML samples, demonstrating that DNTs from a single HD can target an array of AML samples.

FIG. 9. Identifying optimal concentration of DMSO in cryopreservative reagent for freezing of ex vivo expanded DNTs. (A and B) Ex vivo expanded DNTs from healthy donors using methods described herein were frozen in FBS containing 5%, 7.5%, or 10% DMSO. Viability of thawed DNT cells was determined by Annexin V staining on flow cytometry (A) and the cytotoxic function were determined by flow based killing assay against leukemia cell line (B). Horizontal bars represent the mean and error bars represent ±SEM. Unpaired, two-tailed Student's t test was used for statistical analysis.

FIG. 10. Effect of animal serum in freezing media on the viability and anti-leukemic activity of cryopreserved expanded DNT cells. (A and B) Ex vivo expanded DNT cells from a same culture were frozen in freezing media containing same concentration of DMSO with or without animal serum: FBS+7.5% DMSO and Cryostor+7.5% DMSO, respectively. Viability of thawed cells (A) and their anti-leukemic function (B) were determined as described in FIG. 9. Horizontal bars represent the mean and error bars represent ±SEM. Unpaired, two-tailed Student's t test was used for statistical analysis.

FIG. 11. Validating the viability and the function of expanded cryopreserved DNTs. (A and B) Ex vivo expanded DNT cells from a same culture was either frozen or kept in culture: After thawing, viability of thawed cells (A) and their anti-leukemic function (B) were compared with DNTs that were kept in culture without freezing as described in FIG. 9. Horizontal bars represent the mean and error bars represent #SEM. Unpaired, two-tailed Student's t test was used for statistical analysis. (C) Immunodeficient NSG mice were engrafted with primary AML sample, and was treated with PBS or thawed DNTs. Harvested bone marrow cells were stained with anti-human CD45 and CD33 antibody and analyzed on flow cytometry to determine the level of AML engraftment. Each dot represents a mouse, the bar represent the mean, and error bars represent ±SEM. Unpaired, two-tailed Student's f test was used for statistical analysis: *p<0.05.

FIG. 12. Number of DNTs acquired at the end of 14-17 day ex vivo expansion using a previously established research-grade expansion method and the newly established GMP-grade expansion method described herein.

FIG. 13. Ex vivo expansion of DNTs in the presence or absence of plasma (a) or HSA (b) using GMP-expansion method as described herein.

FIG. 14. Ex vivo expansion of DNTs with addition of plasma obtained from autologous (empty symbol) and two allogeneic donors (filled symbols) using GMP-expansion method described herein. (b and c) Viability (b) and anti-cancer activity (c) of ex vivo expanded DNTs against an AML cell line, AML3/OCI using autologous and allogeneic plasma.

FIG. 15. Expansion of pooled donor DNTs. a) Composition of HLA-A2⁻ (HD1) and HLA-A2⁺ (HD2) DNTs at the start (left) and at the end (right) of pooled donor DNT expansion culture determined by HLA-A2 expression pattern. b) Expansion profile of HD1 and HD2 DNTs, and DNTs mixed from HD1 and HD2 at 1:1 at the start of expansion. c) Viability of DNTs at the end of expansion gated on HLA-A2⁺ (left) and HLA-A2⁻ (right) DNTs. d) Purity of HD1, HD2, and HD1 and HD2 mixed DNT cultures at the end of expansion. Numbers represent the frequency of cells at each gate e) Cytotoxicity of HD1, HD2, and mixed DNTs against two AML cell lines, AML3/OCI (left) and MV4-11 (right) at varying effector:target ratio.

FIG. 16. DNTs obtained from HLA-A2⁻ and HLA-A2⁺ donors were pooled and expanded for 20 days. HLA-A2⁻ and HLA-A2⁺ DNTs were isolated at the end of expansion of mixed DNTs and used as effector cells against autologous (filled) and allogeneic (empty) DNTs. HD1 conventional CD4⁺ and CD8⁺ T cells (T_{conv}) stimulated with HD2 DNTs was used as a positive control.

FIG. 17. Efficacy and safety of DNT therapy in combination with PBMC. (A and B) Leukemia-bearing mice were treated with DNT, PBMC, or DNT+PBMC. A) Efficacy of each treatments was assessed by determining the level of leukemia engraftment in bone marrow. B) The level of tissue damage caused by each treatment were blindly assessed by a pathologist as described in FIG. 2. C) Survival of naïve NSG mice treated with xenogenic GvHD-inducing human PBMC with or without DNTs.

FIG. 18. DNT therapy enhances the overall anti-leukemic activity without hampering graft vs. leukemia (GvL) activity mediated by T_{conv} cells. (A and B) Schematic diagram showing the experimental model used to determine the additive anti-leukemic activity of DNT cells when combined with PBMC (A). Flow cytometry plots are representative of bone marrow leukemia engraftment in mice treated with PBMC+PBS and PBMC+DNT. The dot graph shows the summary of leukemia engraftment levels in each treatment groups (B), (C) The effect of DNT infusion on anti-leukemic activity of CD8⁺ T cells determined by comparing ex vivo cytotoxicity of CD8⁺ T cells isolated from mice treated with PBMC+PBS and PBMC+DNT cells against the same leukemia target used for engraftment.

FIG. 19. DNTs obtained from PBMCs instead of whole blood can be expanded with comparable expansion fold, purity, and anti-leukemic function. DNTs were isolated from PBMCs obtained from whole blood or leukapheresis samples. A) Purity of PBMC-derived DNTs expanded for 17 days. B) Comparison of expansion folds between DNTs isolated from PBMC and DNTs obtained from whole blood as previously described¹⁶. C) Comparison of in vitro cytotoxicity of DNT as Isolated from PBMC with those obtained from whole blood against OCI-AML3 and MV4-11.

### DETAILED DESCRIPTION OF THE INVENTION

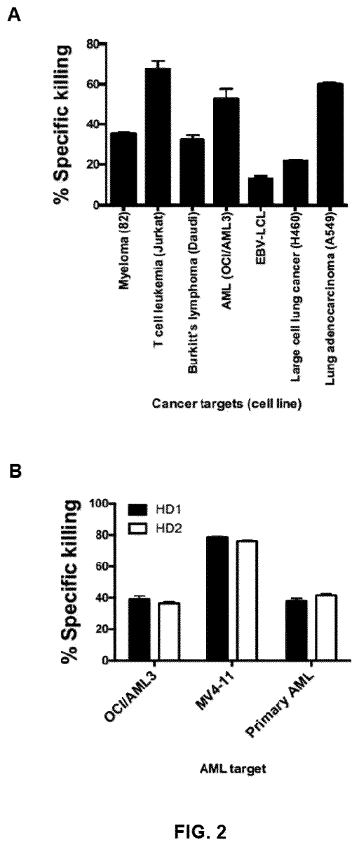

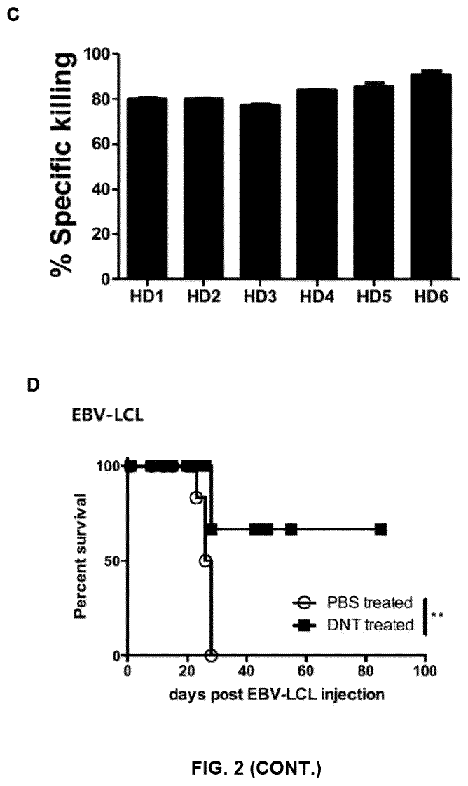

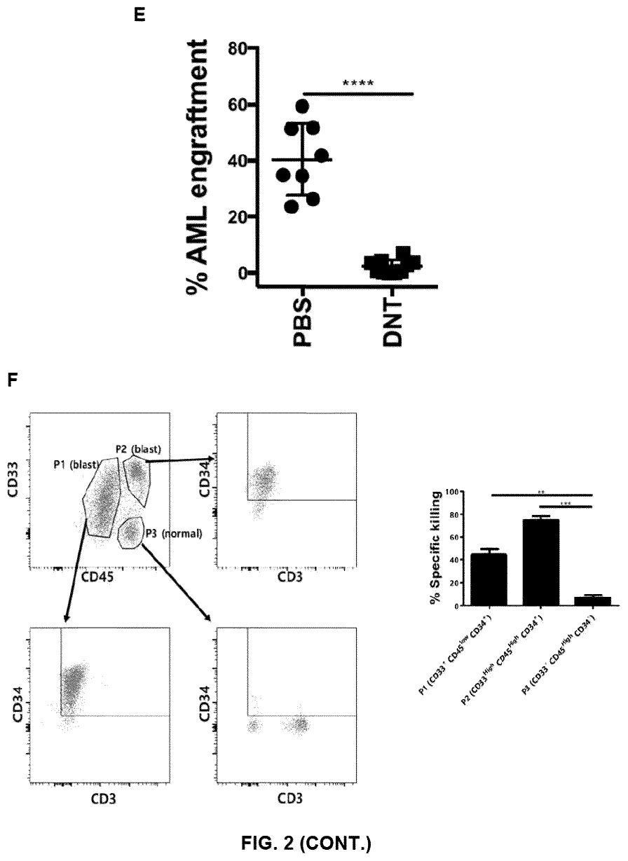

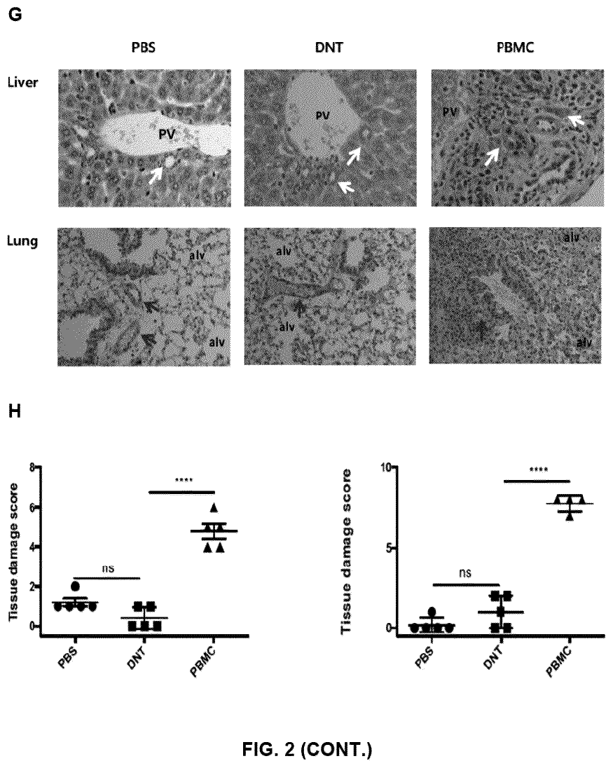

One of major limitations of using allogeneic T cell therapy in the clinic is the risk of GvHD by infused donor cells. Conventional approach in developing allogeneic T cell therapy relies on knocking out/down the TCRα using gene editing or RNA interference technologies, or Immunosuppression.^{17,18} However, we have demonstrated that allogeneic DNTs do not attack normal PBMCs without knocking out its TCR and provide a novel method to overcome the GvHD issue for developing allogeneic off-the-shelf T cell therapy without the need to remove TCR. There is a possibility though that when DNTs are activated against a leukemic target, they may induce cytotoxicity to nearby normal cells. To assess the potential alloreactivity of ex vivo expanded HD DNTs against normal cells during their cancer-targeting activity, in vitro killing assay was conducted using allogeneic DNTs against AML patient PB derived leukemic samples, which contained a mixture of leukemic cells and normal cells defined by CD33, CD34, and CD45 expression pattern (FIG. 2F). Notably, DNTs induced potent cytotoxicity against two leukemic blast population (P1 and P2), but no cytotoxicity was seen against normal cell population (P3; FIG. 2F), demonstrating that even in a single culture, DNTs can selectively recognize and target leukemic blasts and spare normal cells from the same recipient. To further validate this, human AML-bearing mice were treated with PBS, PBMC or DNTs. Consistent with the in vitro finding, a significant anti-cancer activity of DNTs was observed in xenograft models, but DNT-treated mice did not exhibited signs of xenogeneic GvHD, unlike PBMC treated group (FIG. 2G). Liver tissue from PBMC treated mice showed moderate portal lymphocytes infiltration and severe bile duct injury (white arrows). In contrast, DNT-treated mice showed mild portal lymphocyte infiltration but no bile duct injury. In lung, PBMC-treated mice show severe inflammation around vessels (black arrows) and bronchioles (grey arrows), and there are also endothelitis and septal inflammation around alveoli (alv). In contrast, DNT treated mice show no inflammation around vessels and bronchioles, and no endothelitis or septal inflammation around alveoli. Tissue damages seen in histology slides were blindly scored by a pathologist, and scored significantly lower tissue damage score in DNT-treated group than that of PBMC-treated (FIG. 2H),

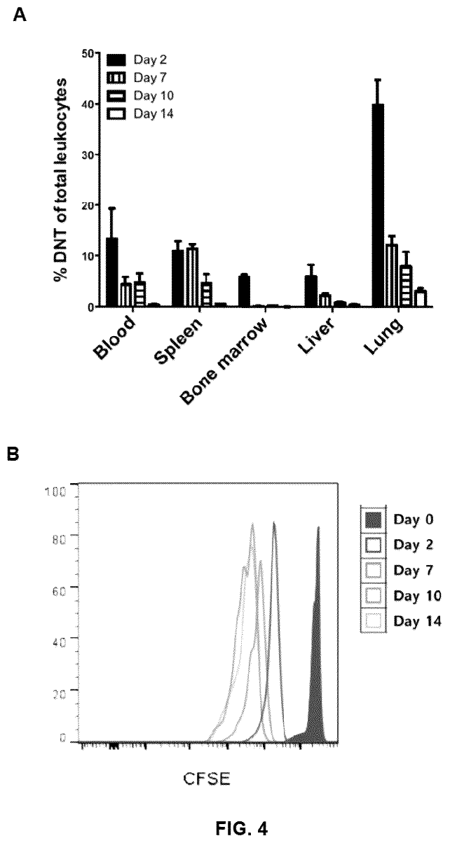

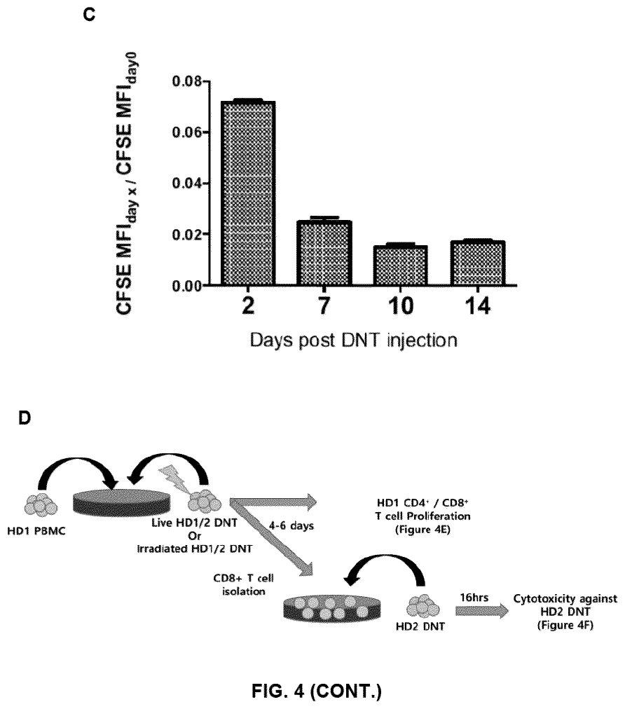

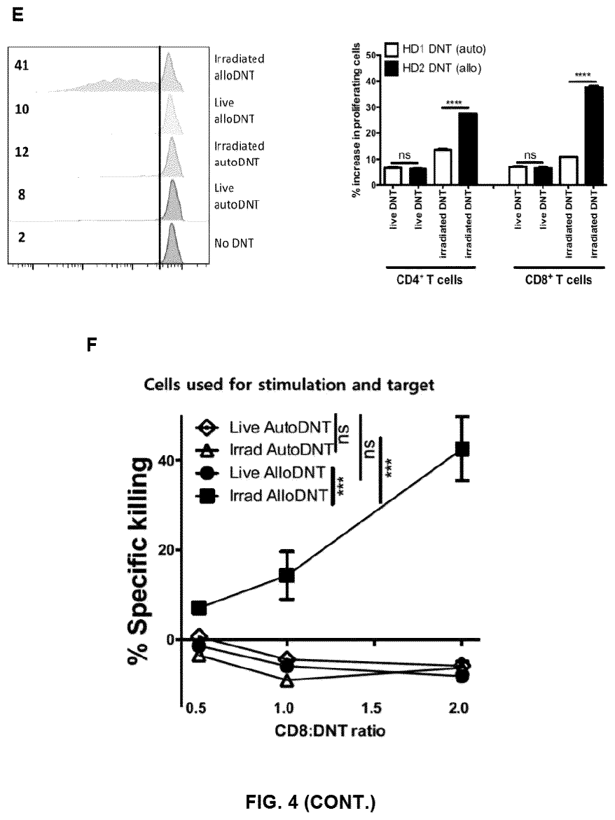

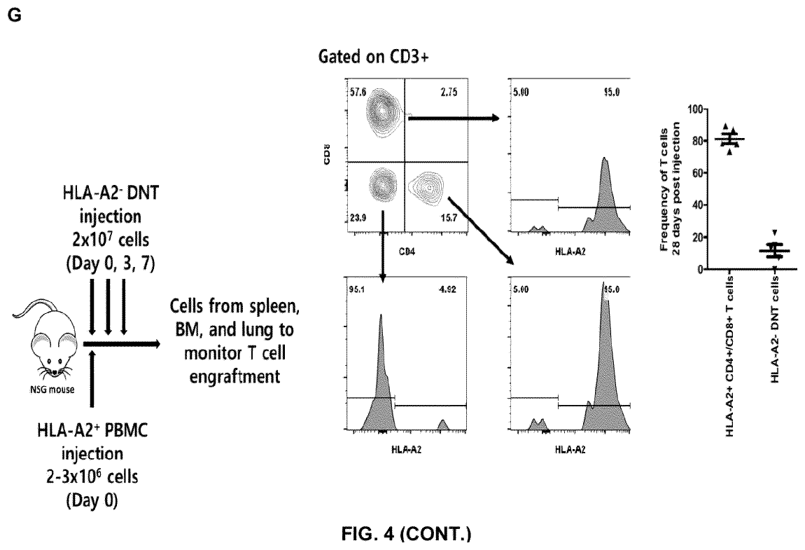

The persistence of infused immune cells has been shown to be correlated with treatment outcomes.¹⁹ Common limitation of allogeneic therapy is the quick rejection of infused cells by the host-immune system through a phenomenon called host-versus-graft (HvG) rejection.^{6,20} Conventional approaches to overcome the HvG issue for allogeneic T cell therapy attempt to disable the MHC-I by knocking out beta 2 microglobulin (beta-2M) chain through gene editing, or rely on immunosuppression.^{20,21} Here, it has surprisingly been determined that clinical-grade DNTs generated using the methods described herein do not induce alloreactivity, and can persist in the presence of allogeneic T cells without the need of gene editing to remove MHC-I, and provide a novel method to overcome the HvG issue for developing allogeneic, off-the-shelf cell therapy without the need to remove MHC-I or MHC-II. FIG. 4D shows the mixed-lymphocyte reaction (MLR) conducted to determine if allogeneic DNTs will induce alloreactivity of recipient's conventional T cells, where HD1 PBMC was cocultured with autologous DNT or allogeneic DNTs from HD2. To determine if allogeneic DNTs carry allo-antigens that can activate conventional T cells, in another group, DNTs were irradiated prior to the MLR. As shown in FIG. 4E, PBMC co-cultured with live or Irradiated autologous DNTs and live allogeneic DNT showed no significant level of proliferation. In contrast, PBMC stimulated with irradiated allogeneic DNTs induced a significant level of proliferation, suggesting that conventional alloreactive T cells are not activated by live DNTs, although DNTs do carry allo-antigens that can be recognized, as shown with irradiated allogeneic DNTs culture. Subsequently, CD8⁺ cytotoxic T cells were isolated from the MLR and used as effector cells against DNTs initially used for stimulation. While those stimulated with autologous DNT or live allogeneic DNTs did not induce cytotoxicity, CD8⁺ T cells stimulated with Irradiated allogeneic DNTs did, supporting the notion that live DNTs do not result in alloreactivity of conventional T cells (FIG. 4F).

To validate this finding in vivo, NSG mice were infused with PBMC from HLA-A2⁺ donor and DNTs from HLA-A2⁻ donor (FIG. 4G). Twenty-eight days post infusion, cells from various tissues of the recipient mice were obtained and analyzed for the frequency of CD4⁺ and CD8⁺ T cells, DNTs and donor CD4+ and CD8+ T cells were identified by HLA-A2 expression. Persistence of HLA-A2⁺ CD4⁺ T cells, CD8⁺ T cells, and HLA-A2⁻ DNTs were detected in the same tissue, demonstrating that allogeneic DNTs can co-persist with conventional T cells. To further investigate the allo-reactivity of engrafted CD8⁺ T cells, HLA-A2⁺ CD8⁺ T cells were subsequently isolated from DNT- and PBMC-treated mice and used as effectors against HLA-A2⁻ DNTs from the same donor origin as used for the xenograft experiment. No significant decrease in DNT cell viability was seen in the presence of isolated HLA-A2⁺ CD8⁺ T cells (FIG. 7), demonstrating that allogeneic CD8⁺ T cells did not develop alloreactivity against DNTs in a xenograft model. Collectively, these data suggest that ex vivo expanded DNTs are resistant to allogeneic immune cell-mediated rejection and provide a foundation to further test the potential of allogeneic DNT as an off-the-shelf ACT that is resistant to HvG rejection in a clinical study.

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells, as in a hematological cancer such as leukemia.

The term “cancer cell” refers a cell characterized by uncontrolled, abnormal growth and the ability to invade another tissue or a cell derived from such a cell. Cancer cells include, for example, a primary cancer cell obtained from a patient with cancer or cell line derived from such a cell. In one embodiment, the cancer cell is a hematological cancer cell such as a leukemic cell or a lymphoma cell.

The term “subject” as used herein includes all members of the animal kingdom including mammals, and suitably refers to humans. Optionally, the term “subject” includes mammals that have been diagnosed with cancer or are in remission. In one embodiment, the term “subject” refers to a human having, or suspected of having, cancer.

In one embodiment, the methods and uses described herein provide for the treatment of cancer. The term “treating” or “treatment” as used herein and as is well understood in the art, means an approach for obtaining beneficial or desired results, including clinical results, Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. In one embodiment, treatment methods comprise administering to a subject a therapeutically effective amount of DNTs as described herein and optionally consists of a single administration, or alternatively comprises a series of administrations.

In one embodiment, the methods and uses described herein involve the administration or use of an effective amount of DNTs. In one embodiment, the methods and uses described herein involve the administration or use of an effective amount of DNTs in combination with allogenic hematopoietic stem cells (HSCs) and/or peripheral blood mononuclear cells (PBMCs). In one embodiment, the methods and uses described herein involve the administration or use of an effective amount of DNTs in combination with lymphocytes such as conventional T cells, In one embodiment, the PBMCs and/or lymphocytes are allogenic cells. As used herein, the phrase “effective amount” or “therapeutically effective amount” means an amount effective, at dosages and for periods of time necessary to achieve the desired result. For example in the context or treating cancer, an effective amount is an amount that for example induces remission, reduces tumor burden, and/or prevents tumor spread or growth of cancer cells compared to the response obtained without administration of the compound. Effective amounts may vary according to factors such as the disease state, age, sex and weight of the animal. The amount of a given compound or population of cells that will correspond to such an amount will vary depending upon various factors, such as the given drug, compound or population of cells, the pharmaceutical formulation, the route of administration, the type of disease or disorder, the identity of the subject or host being treated, and the like, but can nevertheless be routinely determined by one skilled in the art.

In one embodiment, the methods and compositions described herein involve the administration or use of DNTs. DNTs exhibit a number of characteristics that distinguish them from other kinds of T cells. In one embodiment, the DNTs do not express CD4 or CD8. In one embodiment, the DNTs expanded for 10-20 days express CD3-TCR complex and do not express CD4 and CD8. In one embodiment, expanded DNTs are also CD11a+, CD18+, CD10−, and/or TCR Vα24−Jα18−. In one embodiment, expanded DNTs are also CD49d+, CD45+, CD58+ CD147+ CD98+ CD43+ CD66b− CD35− CD36− and/or CD103−.

In one embodiment, the DNTs described herein express one or more surface markers, cytokines and/or chemokines. In one embodiment, the surface markers comprise one or more cytotoxic molecules such as perforin, gramenzymes TRAIL, NKG2D, DNAM-1, NKp30 and/or KIR2DS4, immune co-stimulatory molecules such as CD28, CD27, CD30, GITR, CD40L and/or HVEM, immune co-inhibitory molecules such as TIM-3, LAIR1, NKG2A, CD94, LAG-3, CD160 and/or BTLA, adhesion molecules such as LFA-1, CD44, CD49d and/or CD62L, and/or chemokine receptors such as CXCR3, CCR3, CCR6 and/or CCR9, cytokine receptors such as CD122 and/or CD127.

In one embodiment, the DNT described herein have no or low expression of immune co-inhibitory molecules PD-1, and/or CTLA-4, are resistant to PD-1 and/or CTLA-4 pathway mediated T cell suppression and exhaustion, and/or cancer immune suppression or escape mechanisms.

DNTs as described herein may be obtained using technologies known in the art such as, but not limited to, fluorescent activated cell sorting (FACS).

As used herein, the term “allogenic” refers to cells which are originally obtained from a subject who is a different individual than the intended recipient of said cells, but who is of the same species as the recipient. Optionally, allogenic cells may be cells from a cell culture. In a one embodiment, the DNTs are allogenic cells obtained from a healthy donor. As used herein the terms “healthy donor” (“HD”) refer to one or more subjects without cancer. In one embodiment, the healthy donor is a subject with no detectable cancer cells, such as a subject with no detectable leukemic cells.

In one embodiment, the DNTs and/or allogenic HSCs and/or PBMCs, optionally donor lymphocytes, may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The term “pharmaceutically acceptable” means compatible with the treatment of animals, in particular, humans.

As used herein, “storage medium” refers to any cell culture medium understood by a person skilled in the art for used for the long-term preservation of mammalian cells (vs., for example, an expansion medium). Storage mediums include mediums optimized for the freezing/cryopreservation of cells (Le. freezing medium or cryopreservation medium). Such mediums may contain animal serum (e.g. fetal bovine serum) or may be animal serum-free. Exemplary storage mediums Include FBS with DMSO and Cryostor®.

As used herein, “cryopreservation” refers to the process by which cells, for example T-cells and preferably DNTs, are preserved by cooling to very low temperatures. Such low temperatures are −70° C. to −90° C., preferably about −80° C. using −80° C. freezer, solid carbon dioxide or −196° C. using liquid nitrogen and are utilized to slow/stop any enzymatic or chemical activity which might cause damage to the cells. Cryopreservation methods seek to reach low temperatures without causing additional damage caused by the formation of intracellular ice crystals during freezing.

Methods for Producing a Population of DNTs, Expanding Cryopreservable Off-the-Shelf DNTs and/or Cryopreserving Expanded DNTs for Clinical Use

In one embodiment, there is provided a method of expanding a population of double negative T cells (DNTs) ex vivo. In one embodiment, the method is for expanding a population of cryopreservable off-the-shelf DNTs ex vivo. In one embodiment, the method comprises:

a) providing a sample population of DNTs, wherein the sample population of DNTs comprises DNTs from one or more donors; and

b) culturing the sample population of DNTs in a culture media to produce an expanded population of DNTS.

In one embodiment, the sample population of DNTs comprises DNTs from two or more donors. In one embodiment, culturing the sample population of DNTs in the culture media produces an expanded population of DNTs, optionally an expanded population of DNTs with more than 80% purity.

In one embodiment, the method comprises culturing the DNTs for at least 5 days, 8 days or 10 days, optionally between 5 days and 20 days. In one embodiment, the DNTs are cultured for between about 8 days and 17 days. In one embodiment, the method comprises culturing the DNTs for at least 5 days, at least 8 days, at least 10 days, at least 12 days, at least 14 days, at least 17 days, at least 20 days, or at least 25 days, optionally between 10 days and 20 days.

As shown in the Examples, It has surprisingly been determined that DNTs from multiple donors do not exhibit alloreactivity against one another. Accordingly, in one embodiment the DNTs from different donors are not alloreactive against one other during expansion.

In one embodiment, allogenic DNTs from two or more donors are combined prior to being expanded ex vivo. In one embodiment, allogenic DNTs from two or more donors are expanded ex vivo separately prior to being combined to form a population of DNTs.

In one embodiment, the culture media is animal serum-free media. In one embodiment, the culture media comprises AIM-V, GT-T551, Stemline T cell Expansion Medium, Immunocult-XF T cell Expansion Medium, Human StemXVivo, Serum-Free Human T cell Base Media, CTS T-cell Expansion SFM, Prime-XV T cell expansion XSFM, or an equivalent animal-serum free human T-cell expansion media. In one embodiment, the culture media is GMP-compliant.

In one embodiment, the culture media further comprises human blood-derived components, plasma, serum, or HSA, optionally human plasma. In one embodiment, the human blood-derived components and DNTs may be from the same individual i.e. autologous to the sample population of DNTs. Remarkably, as shown in the Examples the DNTs may be expanded using human blood-derived components that is allogenic to the sample population of DNTs. For example, in one embodiment the plasma comprises pooled plasma from one or more donors, optionally two or more donors. In one embodiment, the concentration of human blood-derived components in the culture media is between 1-20%, optionally between about 2% and 15%.

In one embodiment, the culture media comprises soluble anti-CD3 antibody, IL-15, IL-7 and/or IL-2. In one embodiment, the culture media comprise recombinant or exogenous IL-2, IL-15, IL-7, IFNgamma, an anti-4-1BB, anti-CD28, anti-OX40, anti-ICOS, anti-CD40, recombinant CD83, MIP-1a, IL-6, IL-8, IL-21, Jq1 inhibitor and/or anti-CD3. In one embodiment, the culture media does not comprise exogenous IL-4. For example, in one embodiment, the culture media comprises between about 50 and 500 or between about 50 and 800 IU/ml IL-2 and/or between about 0.05 and 1.0 ug/ml anti-CD3. In one embodiment, the method comprises adding anti-CD3 antibody and/or IL-2 to the culture media.

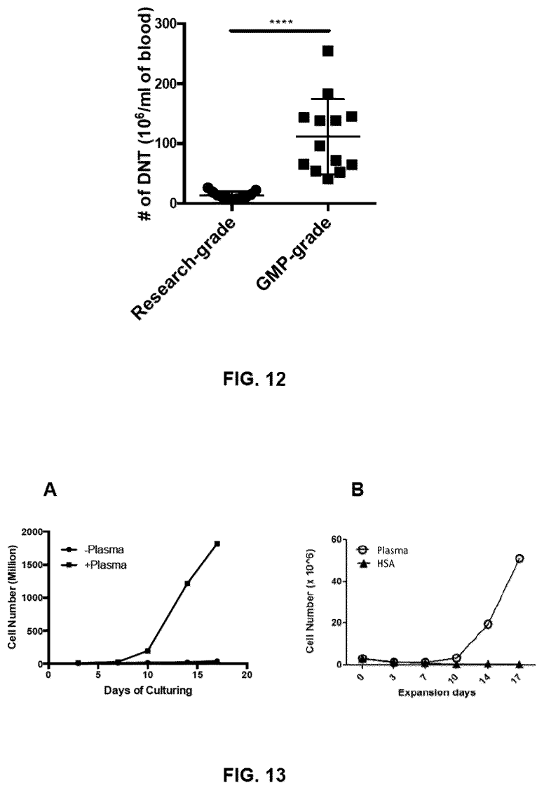

As shown in FIG. 12, the methods described herein are able to produce a significant expansion of DNTs from human samples. In one embodiment, the population of DNTs comprises DNTs from peripheral blood and the expanded population of DNTs yields at least 0.1, 0.2, 0,5, 0.8 or 1.0×10⁸ DNTs per milliliter of peripheral blood. The methods described herein also produce populations of

DNTs with a relatively high level of purity. For example, in one embodiment, the expanded population of DNTs comprises or consists of at least 50, %, 60%, 70%, 75%, or 80% DNTs, optionally at least 85% or 90% DNTs. In one embodiment, the expanded population of DNTs comprises at least 80% DNTs, optionally at least 85% or 90% DNTs.

In one embodiment, the method comprises splitting the cells in order to maintain a healthy and expanding cell population. In one embodiment, the method comprises splitting the cells to maintain a cell population above 0.1 million per ml of the culture media and below 4 million per ml of the culture media.

Various sources of a sample population of DNTs may be used to produce or expand a population of DNTs as described herein. For example, in one embodiment, wherein the sample population of DNTs comprises or consists of DNTs from peripheral blood, leukopheresis, Leukopak, bone marrow and/or cord blood samples.

In one embodiment, DNTs described herein are genetically modified. For example, in one embodiment, the DNTs are recombinant cells that have been modified to express one or more exogenous proteins. In one embodiment, the DNTs are genetically modified to enhance their anti-tumor activities and to reduce the risk to recipients.

In another embodiment, the DNTs are not genetically modified. In one embodiment, the DNTs are not genetically modified to reduce or prevent expression of TCR and/or MHC-I/II. In one embo

### Methods for the Cryopreservation of DNTs

In one embodiment, there is provided a method for cryopreserving double negative T cells (DNTs). In one embodiment the method comprises:

a) Te-suspending a population of DNTs in a storage medium;

b) adding DMSO to the storage medium to a final concentration of between about 2.5% and about 15% DMSO; and

c) cryopreserving the population of DNTs in the storage medium at a temperature less than −70° C.

In one embodiment, the method comprises re-suspending a population of DNTs expanded using a method as described herein in the storage medium. In one embodiment, the method further comprises expanding a population of DNTs using a method as described herein prior to re-suspending the population of DNTs in the storage medium.

In one embodiment, the method comprises cryopreserving the population of DNTs in the storage medium at a temperature between −70° C. to −90° C., preferably about −80° C.

In one embodiment, the population of DNTs has been expanded ex vivo prior to cryopreserving the cells. For example, the DNTs may be expanded ex vivo prior to cryopreserving the DNTs using a method for expanding a population of DNTs ex vivo as described herein.

In one embodiment, the cells are expanded ex vivo for between 5 and 25 days, optionally between about 8 and 14 days, or about 10 days prior to cryopreserving the cells. In one embodiment, the cells are expanded for between about 8 and 20 days prior to cryopreserving the cells.

In one embodiment, the method for cryopreserving the population of DNTs described herein involves the addition of DMSO. In a preferred embodiment, DMSO is added dropwise to the storage medium. In one embodiment, the final concentration of DMSO is from about 3% to 15%, 4% to 10%, or from about 5% to about 8.5%. In one embodiment, the final concentration of DMSO is from about 7% to 8%, optionally about 7.5%.

In one embodiment, DMSO is added to the storage medium such that the rate of increase of the concentration of DMSO in the storage medium is controlled.

In one embodiment, wherein the DMSO prior to being added to the storage medium is at a concentration of about 10% to about 20%, optionally at a concentration of about 10%, about 15% or about 20%.

In one embodiment, DNTs are at a final concentration in the storage medium of between about 2.5×10⁷ and about 2.5×10⁸ cells/ml optionally between about 5-10×10⁷ cells/ml.

In one embodiment, the storage medium in contact with the DNTs is cooled. For example, in one embodiment, the population of DNTs is resuspended in storage medium cooled to less than 10° C. but not frozen, optionally wherein the storage medium is cooled to about 8° C., 6° C., 4° C., or 2° C.

In one embodiment, the method further comprises after step b), but before step c), storing the population of DNTs at a temperature of about 1° C. to about 7° C. In one embodiment, the method comprises storing the population of DNTs for between about 2 minutes and 20 minutes, optionally about 5 minutes, about 10 minutes or about 15 minutes.

As set out in the Examples, the choice of storage medium can impact the viability and/or activity of the DNTs. In one embodiment, the storage medium comprises animal serum, optionally fetal bovine serum. In one embodiment, the storage medium is animal serum free, preferably Cryostor™.

Optionally, cells cryopreserved according the method described herein may then be stored at a temperature less than −130° C., optionally in liquid nitrogen. In one embodiment, the method comprises storing the population of DNTs at the temperature between −70° C. to −90° C. for at least 8 hours, at least 10 hours, at least 12 hours or at least 16 hours prior to storing the cryopreserved cells at the temperature less than −130° C.

### Pharmaceutical Compositions and Populations of Cells

In one embodiment, there is provided a population of DNTs that has been expanded and/or cryopreserved according to a method as described herein. In one embodiment, the population of DNTs is for use in the treatment of cancer. Also provided is a population of DNTs that has been expanded, and/or cryopreserved according to a method as described herein in combination with a population of hematopoietic stem cells (HSCs), wherein the DNTs and HSCs are from the same donor or different donors. In one embodiment, DNTs in combination with allogeneic HSCs showed enhanced anti-cancer activity while DNTs also reduces GvHD from allogeneic HSCs. Also provided is a population of DNTs that has been expanded, and/or cryopreserved according to a method as described herein in combination with a population of PBMCs, optionally a population of lymphocytes such as conventional T cells, wherein the DNTs and PBMCs are from the same donor or different donors. In one embodiment, the use of DNTs in combination with allogeneic PBMCs reduces GvHD from allogeneic PBMCs.

In one embodiment, the DNTs from a single expansion of DNTs from one or more donors and are for use or administration In one or more subjects for the treatment of cancer or for use or administration in one or more subjects for multiple treatments of cancer. In one embodiment, the DNTs are from a single expansion of DNTs from two or more donors and are for use or administration in one or more subjects for the treatment of cancer or for use or administration in one or more subjects for multiple treatments of cancer. Also provided is a method of treating cancer in a subject in need thereof, comprising administering to the subject an effective amount of a population of DNTs and optionally HSCs or PBMCs as described herein. Also provided is the use of a population of DNTs and optionally HSCs or PBMCs as described herein for use in the preparation of a medicament, optionally a medicament for the treatment of cancer.

### Methods and Uses of Allogenic DNTs for the Treatment of Cancer

As shown in the Examples, formulations comprising allogenic DNTs from different donors have surprisingly been demonstrated to be useful for the treatment of cancer.

In one embodiment, there is provided a method of treating cancer in a subject in need thereof, comprising administering to the subject an effective amount of a population of double negative T cells (DNTs), wherein the population of DNTs comprises allogenic DNTs from one or more healthy donors (HDs). In one embodiment, the population of DNTS comprises allogenic DNTs from two or more HDs. Also provided is the use of an effective population of DNTs comprising allogenic DNTs from one or more donors or two or more donors for the treatment of cancer.

In one embodiment, the allogenic DNTs have been expanded ex vivo, optionally using a method as described herein. In one embodiment, allogenic DNTS from the two or more donors are combined prior to being expanded ex vivo. In another embodiment, allogenic DNTs from two or more donors are expanded ex vivo separately prior to being combined to form the population of DNTs. In one embodiment, the one or more donors are one or more subjects without cancer,

The populations of allogenic DNTs described herein exhibit a number of characteristics desirable for in vivo use for the treatment of cancer. In one embodiment, DNTs from different donors are not alloreactive against each other in a population of DNTs. In one embodiment, the population of DNTs is resistant to allogenic immune cell-mediated rejection in the subject in vivo.

In one embodiment, the DNTs persist in vivo in the subject for at least 10 days. In one embodiment, the population of DNTs persists in the subject for at least 2 weeks, at least 3 weeks, or at least 4 weeks. In one embodiment, the population of DNTs is not cytotoxic against normal cells in vivo.

In one embodiment, the population of DNTs has been cryopreserved prior to administering the population of DNTs to the subject, optionally by using a method for cryopreserving DNTs as described herein. In one embodiment, the population of DNTs has been cryopreserved without lose of viability and/or function. For example, in one embodiment, the population of DNTs can be cryopreserved for at least 10 days, 30 days, 60 days, 100 days, 300 days, 400 days or 600 days without loss of viability and/or function for the treatment of cancer.

In one embodiment, the population of DNTs is not genetically modified prior to their use or administration for the treatment of cancer. For example, in one embodiment the DNTs are not genetically modified to reduce or prevent expression of TCR and/or MHC-I/II. In one embodiment, the subject is not administered Immunosuppression therapy prior to or during the administration of the population of DNTs for the treatment of cancer. In another embodiment, the subject is administered immunosuppression therapy prior to or during the administration of the population of DNTs for the treatment of cancer.

As shown in the Examples, DNTs described herein may be used in combination with allogenic hematopoietic stem cells (HSCs) and/or peripheral blood mononuclear cells (PBMCs) for the treatment of cancer. In one embodiment, the PBMCs are lymphocytes, optionally conventional T cells. Accordingly, in one embodiment, the methods described herein include administering to a subject in need thereof DNTs and a population of cells comprising HSCs. in one embodiment, the methods described herein include administering to a subject in need thereof DNTs and population of cells comprising PBMCs. Also provided is the use of a population of DNTs as described herein in combination with a population comprising allogenic HSCs for the treatment of cancer. Also provided is the use of a population of DNTs as described herein in combination with a population comprising allogenic PBMCs for the treatment of cancer.

In one embodiment, the DNTs are allogenic DNTs from a plurality of healthy donors, optionally wherein the DNTs are expanded according to a method described herein.

In one embodiment, the subject is not administered immunosuppression therapy prior to or during the administration of the population of DNTs. In one embodiment, the DNTs described herein are for use or administration in a subject in the absence of immunosuppression therapy.

In one embodiment, the population of DNTs is from a single expansion of DNTs from one or more donors, optionally two or more donors, and for use or administration to a single cancer patient or to a plurality of cancer patients.

In one embodiment, the population of DNTs is from a single expansion of DNTs from one or more donors and is for use or administration to multiple different subjects for the treatment of cancer.

In one embodiment, the DNTs are allogenic DNTs that have been expanded and/or cryopreserved ex vivo, optionally according to a method described herein.

In one embodiment the DNTs are for use or administration to the subject at the same time as the HSCs and/or PBMCs or at different times. For example, in one embodiment the DNTs are for use or administration to the subject within 1 hours, 2 hours, 4 hours, 8 hours, 12 hours, 1 day, 2 days, 3 days, 4 days, 5 days, 6 days, 1 week, 10 days, 2 weeks, 3 weeks, 4 weeks, or more of the use or administration of the HSCs and/or PBMCs. In one embodiment, the combination of DNTs and HSCs and/or PBMCs is for the treatment of myelodysplastic syndrome, non-Hodgkin lymphoma, Hodgkin lymphoma, multiple myeloma, or leukemias in the subject.

In one embodiment, the HSCs are from peripheral blood, leukapheresis, bone marrow or cord blood. In one embodiment, the allo-HSCs are mobilized using G-CSF. In one embodiment, the DNTs and HSCs are from the same donor. In one embodiment, the DNTs and HSCs are from different donor and optionally are allogenic DNTs and HSCs for use in the treatment of cancer.

In one embodiment, the PBMCs are lymphocytes, optionally conventional CD4+ CD8+ T cells. In one embodiment, the DNTs are for use or administration to the subject at the same time as the PBMCs or at different times.

In one embodiment, the methods and uses described herein include inhibiting immune co-inhibitory molecules using anti-TIM3, anti-NKG2A, anti-LAIR1, anti-CD94, anti-LAG3, anti-CD160 and/or anti-BTLA antagonistic agents, and/or through enhancing immune co-stimulatory molecules using anti-CD28, anti-CD27, anti-GITR, anti-CD40L, anti-HVEM and/or anti-CD30 agonistic agents. In one embodiment, there is provided a method to enhance activity of DNTs, the method comprising inhibiting immune co-inhibitory molecules using anti-TIM3, anti-NKG2A, anti-LAIR1, anti-CD94, anti-LAG3, anti-CD160 and/or anti-BTLA antagonistic agents, and/or through enhancing immune co-stimulatory molecules using anti-CD28, anti-CD27, anti-GITR, anti-CD40L, anti-HVEM and/or anti-CD30 agonistic agents. In one embodiment, the method comprises the use or administration of anti-CD3 to enhance the anti-cancer activity of DNTs.

Accordingly, in one embodiment, the methods and uses described herein comprise the use or administration of anti-CD3 anti-TIM3, anti-NKG2A, anti-LAIR1, anti-CD94, anti-LAG3, anti-CD160 and/or anti-BTLA antagonistic agents, and/or anti-CD28, anti-CD27, anti-GITR, anti-CD40L, anti-HVEM and/or anti-CD30 agonistic agents. In one embodiment, the methods and uses described herein for the treatment of cancer further comprise the use or administration of an antibody to CD3. In one embodiment, the antibody to CD3 is for use or administration to the subject at the same time or at different times as the use or administration of the DNTs.

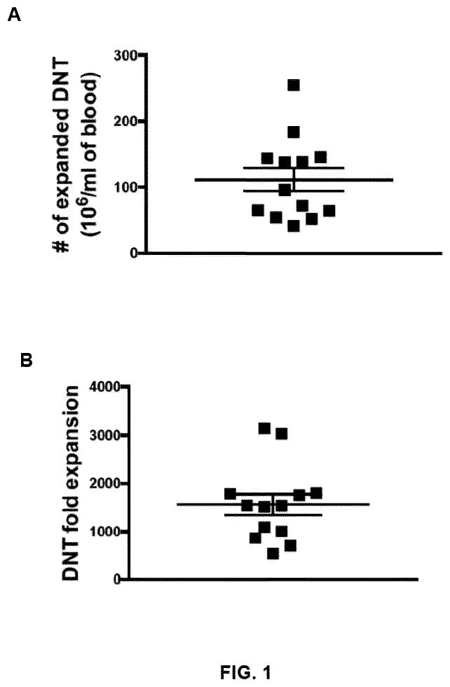

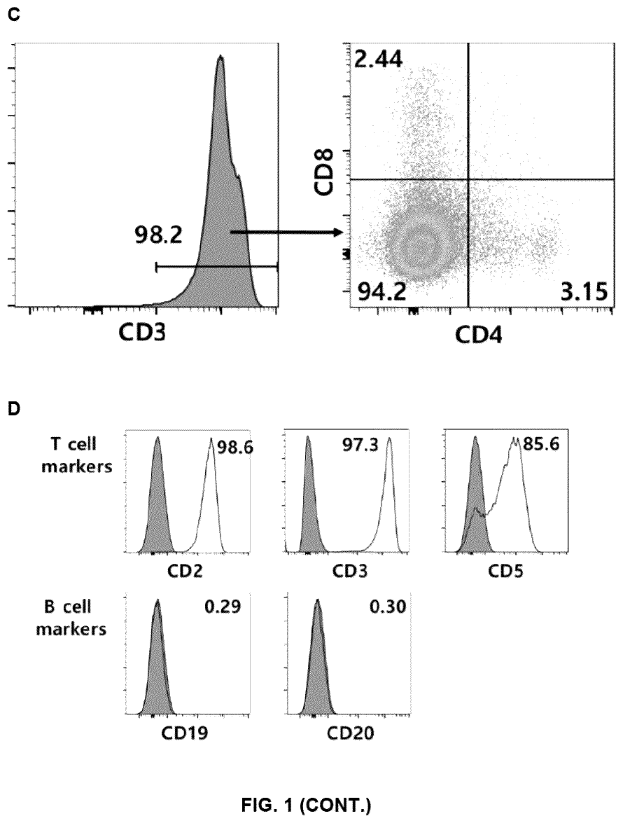

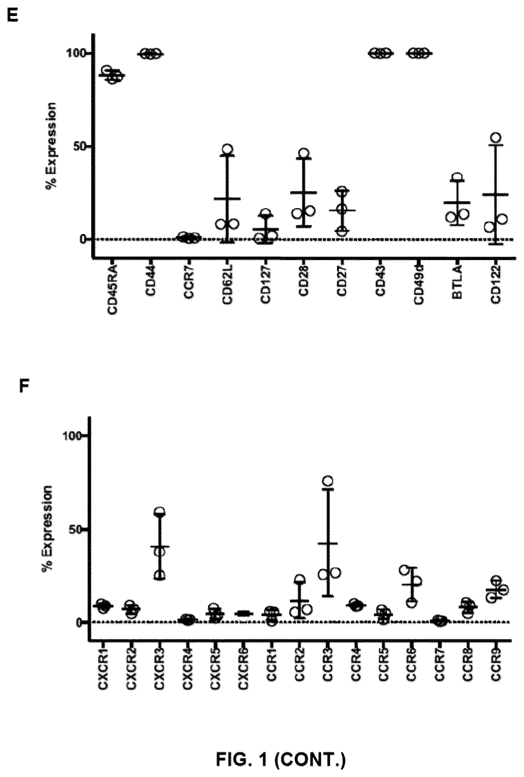

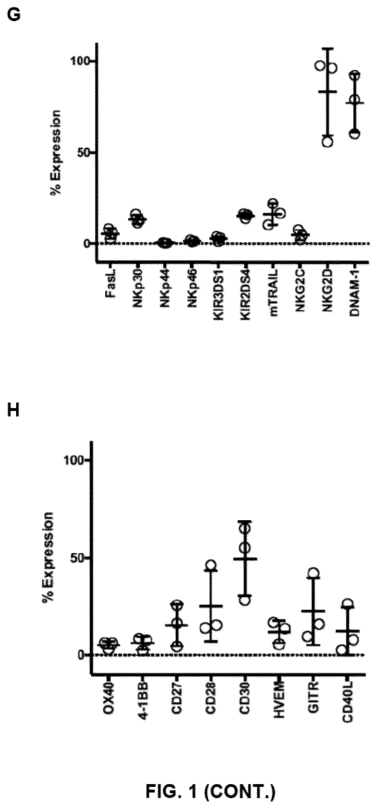

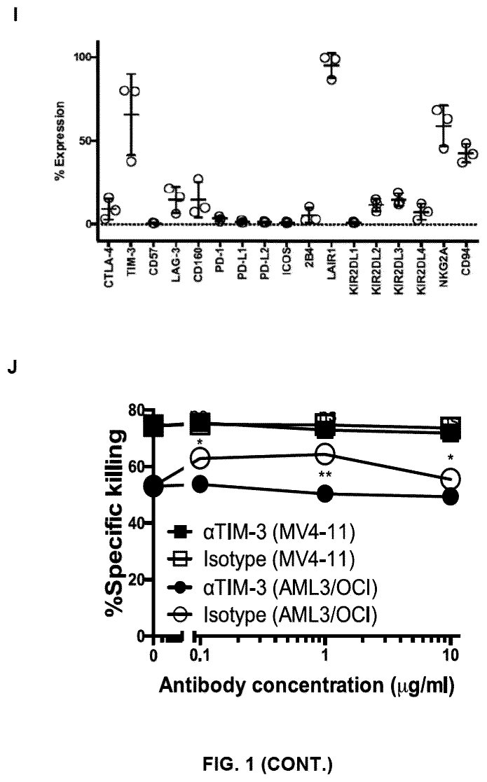

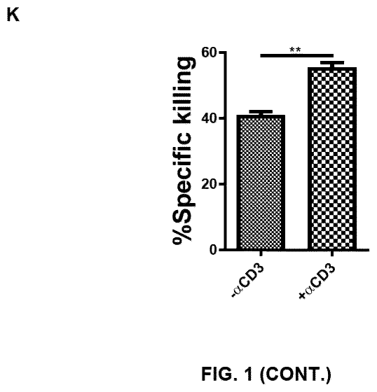

As shown in FIGS. 1J and 1K, the addition of TIM-3 or CD3 antibody modulated the level of killing mediated by DNTs against AML3/OCI. Combination therapy using DNTs and antibodies against molecules expressed on DNTs to improve their function is therefore expected to improve the therapeutic applications of DNTs for the treatment of cancer. In one embodiment, the antibodies are for use or administration at the same time as the DNTs or at different times. For example, in one embodiment the DNTs are for use or administration to the subject within 1 hours, 2 hours, 4 hours, 8 hours, 12 hours, 1 day, 2 days, 3 days, 4 days, 5 days, 6 days, 1 week, 10 days, 2 weeks, 3 weeks, 4 weeks, or more of the use or administration of the antibodies.

Also provided is a method to modulate tissue trafficking and homing of DNTs. In one embodiment, the method comprises inducing or delivering adhesion ligands/receptors to CD44, CD49d and/or CD62L and/or chemokines to CXCR3, CCR3, CCR6 and/or CCR9 at desired target tissues and locations.

The following non-limiting examples are illustrative of the present disclosure:

### Example 1: Allogeneic Double Negative T Cells as an Off-the-Shelf Adoptive Cellular Therapy for Cancer

Adoptive T cell therapy is a practical treatment option for cancer patients. However, with an increase in the clinical-use of adoptive cellular therapy (ACT), its limitations, including high treatment costs and technical requirements, are becoming apparent and are restricting the wide clinical-use of ACT.²² Off-the-shelf allogeneic ACT has several advantages including lower treatment cost, reliable supply of cellular products and easy accessibility, but several requirements must be met before its clinical applications.^{12,20} This example describes a straight-forward and easily applicable method to expand cryopreservable clinical-grade double negative T cells from healthy donors that fulfills the requirements of an off-the-shelf ACT: a therapy that targets various cancer types without apparent off-tumor toxicity; can overcome host-versus-graft reaction and achieve sufficient persistence; and is storable. Remarkably, the use of DNTs as described herein represents a T cell therapy that can be used as an off-the-shelf therapy without any genetic modification.

### Materials and Methods

### Ex Vivo Expansion of DNTs Under GMP Conditions

DNT expansions were done as previously described²⁰ under GMP conditions with some modifications. Briefly, CD4⁺ and CD8⁺ cell-depleted PBMCs were cultured on anti-CD3 antibody-coated plates (GMP grade OKT3; Miltenyi) for 3 days in serum-free media (AIM-V (ThermoFisher) or GT-551 (Takara Bio)) with 250 IU/ml of IL-2 (Proleukin, Novartis Pharmaceuticals, Canada); soluble anti-CD3 antibody and IL-2 were added to the cultures. The purity of DNTs was assessed on days 0 and 10 of expansion as well as after harvesting before use for subsequent experiments. DNT purity was measured by staining cells with fluorochrome-conjugated anti-human CD3, -CD4, -CD8, and -CD56 antibodies and flow cytometry analysis. For validation runs, DNTs were expanded at the Philip S. Orsino Cell Therapy Facility at Princess Margaret Cancer Centre or at Sunnybrook Research Institute GMP facility. To test for sterility, mycoplasma, and endotoxin, expanded DNT products were sent to Mount Sinai Hospital, WuXiApp Tech, and Princess Margaret Cancer Centre, respectively.

### Flow Cytometry-Based In Vitro Killing Assay

For non-adherent cancer cells, DNTs were co-cultured with target cells for 2-4 hours, cells were then stained with anti-human CD3 (HIT3a), CD33 (WM53), CD45 (HI30), and CD34 (561) antibodies, Annexin V, and 7AAD (all from BioLegend), and analyzed using flow cytometry. Specific killing was calculated by

$$\frac{\% {AnnexinV}_{with DNT}^{+} - \% {AnnexinV}_{with out DNT}^{+}}{100 - \% {AnnexinV}_{without DNT}^{+}} \times 100.$$

For adherent cancer cells, cell lines were labelled with DiO (Invitrogen) and co-cultured with DNTs for 14 hr. All cells were collected after incubation in 0.25% trypsin-EDTA solution and stained with TO-PRO-3 (Life Technologies). Cell suspensions were analyzed by flow cytometry to determine specific lysis of labelled target cells. Specific killing was calculated by:

$$\frac{\% {DiO}^{+} TO PRO - {3}_{with DNT}^{+} - \% {DiO}^{+} TO PRO - {3}_{without DNT}^{+}}{100 - \% {DiO}^{+} TO PRO - {3}_{without DNT}^{+}} \times 100.$$

### Antibodies and Flow Cytometry

The following anti-human antibodies were used for cell staining: CD3-FITC or -PECy7, CD4-FITC or -PE, CD8-FITC or -PE, CD33-APC or -PECy5, CD56-PE, iNKT TCR (Vα24-Jα18 TCR)-APC, and Annexin V-FITC or -Pacific Blue and were purchased from BioLegend. Data acquisition was performed using either a BD Accuri C6 Flow cytometer (BD Bioscience) or an Attune NXT cytometer (ThermoFisher). Flow cytometry data were analyzed using FlowJo software (Tree Star, Inc.).

### High-Throughput Flow Cytometry Screening

Ex vivo expanded DNTs were prepared for flow-cytometry based high-throughput screening as described previously.²³ Briefly, expanded DNTs were spun down and treated with FcX TrueStain (Biolegend) in PBS containing 0.5% BSA for 10 mins followed by staining with anti-CD3 PE-Cy7 antibody. Subsequently, cells were sent to Princess Margaret Genomics Centre, where cells were stained with antibodies against 385 different cell surface molecules followed by staining with a viability dye, DAPI, prior to being analyzed by flow cytometry. Intracellular staining of CTLA-4 was performed using the protocol described herein. Data were analyzed using FlowJo software (Tree Star, Inc.).

### Cryopreservation of DNTs

Day 7-20 ex vivo expanded DNTs were re-suspended in 4° C. CryoStor2 (StemCell Tech.) containing 2% clinical-grade DMSO (StemCell Tech.), followed by adding an appropriate volume of CryoStor10 (StemCell Tech.) containing 10% DMSO to make the final concentration of DMSO at 7.5% and cell concentration at 5-10×10⁷ cells/ml. Cells were transferred to −80° C. freezer in CoolCell™ (Fisher Scientific) to allow slow temperature decrease. Frozen cells were transferred to liquid nitrogen the next day for longer storage.

To thaw DNTs, cells taken from liquid nitrogen were thawed in a 37° C. water- or bead-bath and serum was added at 20× the volume of cells, Cells were centrifuged at 300×g for 10 min. Pellets were resuspended at 10⁶ cells/ml with IL-2 and anti-CD3 (OKT3) antibody containing serum free culture media for further use,

### Mixed Lymphocyte Reaction

CFSE-labeled or unlabeled PBMC obtained from healthy donors were co-cultured with live or irradiated expanded DNTs from autologous or allogeneic donor at 2:1 PBMC to DNT ratio for 4-6 days. Percent proliferating cells based on CFSE dilution was determined by flow cytometry. Percent increase in proliferation was calculated by:

$$\frac{\% {Proliferated}_{with DNT} - \% {Proliferated}_{without DNT}}{100 - \% {Proliferated}_{without DNT}} \times 100.$$

To determine allo-reactivity, CD8⁺ T cells were Isolated using a CD8-positive selection kit (StemCell Tech.) and isolated CD8⁺ T cells were co-cultured with DNTs at 4:1 CD8:DNT ratio for 4 to 14 hours. The cells were then stained with Annexin V and anti-CD8 antibody and analyzed by flow cytometry.

### Xenograft Models

For all xenograft experiments, NOD.Cg-Prkdc^{scid} Il2rg^{Im1Wji}/SzJ (NSG) mice (Jackson Laboratories, Bar Harbor, ME) maintained at the University Health Network (UHN) animal facility were used. To characterize persistence of DNTs, 8-12 week old female mice were irradiated (250 cGy) 24 hours prior to a single injection of 5 μM CFSE-labelled 2×10⁷ DNTs. Cells from the bone marrow, spleen, liver, lungs, and peripheral blood were harvested on days 2, 7, 10, and 14, and the frequency of DNTs and CFSE dilution were determined by flow cytometry. To determine anti-cancer activity in vivo, irradiated NSG mice were infused with 1-5×10⁶ MV4-11 or EBV-LCL cells through tail vein injection, 1-3×10⁷ DNTs were injected Intravenously on days 3, 6, and 10 post-cancer cell injection. MV4-11 Infused mice were sacrificed two weeks after the last DNT injection, and the engraftment of MV4-11 in the bone marrow was determined using flow cytometry as described previously²⁰. EBV-LCL infused mice were euthanized when their body weight decreased by 20%. To assess the tissue damage, MV4-11 bearing mice were infused with DNTs, as described above, or PBMCs as a positive control. The liver and lung tissues were harvested and fixed in 10% formalin overnight and sent to Pathology Research Program Laboratory (Toronto General Hospital) for haemotoxylin and eosin (H&E) staining. The H&E stained histology slides were blindly scored by a pathologist for tissue damage following a previously described scoring method²⁴ with some modifications. The modified scoring method used is described herein. To determine the allo-reactivity of DNTs, mice were infused with 2-3×10⁶ HLA-A2⁺ PBMC on day 0 and HLA-A2⁻ DNTs on day 0, 3, and 6. Four weeks post-infusion, cells from bone marrow, spleen, and lung were analyzed by flow cytometry to monitor the engraftment level of human T cells. In all experiments, rIL-2 (Proleukin) was administered (10⁴ IU/mouse) intraperitoneally at the time of DNT infusion and weekly after the last DNT injection until euthanization.

### Statistical Analysis

All graphs and statistical analysis were generated using GraphPad Prism 5. Student's t-test and linear regression test were used. *p<0.05; **p<0.01; ***p<0.001; ****p<0.0001 indicate significance between experimental and control values. Error bars represent ±SEM or SD as indicated.

### Human Samples and Study Approval

Human blood was collected from healthy adult donors after obtaining written informed consent and used according to UHN Research Ethics Board (05-0221-T). Animal studies were approved by the Institutional Animal Care Committee of UHN (AUP: 741,22) and carried out in accordance with the Canadian Council on Animal Care Guidelines.

### Antibodies, Flow Cytometry and ELISA

The following anti-human antibodies were used for cell staining: CD3-FITC or -PECy7, CD4-FITC or -PE, CD8-FITC or -PE, CD34-FITC or -PE, and CD33-APC or -PECy5 were purchased from BioLegend. Data acquisitions were performed using either BD Accuri C6 Flow cytometry (BD Bioscience) or LSRII (BD Biosciences) Flow cytometers and data were analyzed using FlowJo software (Tree Star, Inc.).

### Cell Lines

Cell lines, AML3/OCI, MV4-11, Jurkat, Daudi, H460, and A549 were obtained from ATCC KG1a, EBV-LCL was obtained from The Center for Applied Genomics at The Hospital for Sick Children. AML3/OCI were cultured in alpha-MEM supplemented with 10% fetal bovine serum (FBS), EBV-LCL, Jurkat and Daudi were cultured in RPMI-1640 supplemented with 10% FBS, MV4-11 was cultured in IMDM supplemented with 10% FBS, H460 and A549 were maintained in DMEM/F12 supplemented with 10% FBS. All cell lines were incubated at 37° C. in 5% CO₂.

### Intracellular Staining of CTLA-4

Ex vivo expanded DNTs surfaced stained with anti-human CD3, CD4, and CD8 antibodies, and fixed and permeabilized using intracellular fixation and permeabilization kit (eBioscience). Permeabilized cells were stained with anti-CTLA-4 antibody (Clone L3D10) for 30 mins at 4° C. After washing, cells were analyzed by flow cytometry.

### GvHD-Model Tissue Damage Scoring

Mice treated with PBS, DNT, or PBMC were sacrificed and liver and lung tissues harvested, fixed in 10% formalin, and H&E stained. Liver and lung histology slides were blindly scored by a pathologist following the scoring charts below:

TABLE 1
Scoring charts for GvHD-model tissue damage

| | | | |
|---|---|---|---|
| 0 | 1 | 2 | 3 |

Liver GVHD scoring

| | | | | |
|---|---|---|---|---|
| Portal | Absent | Mild | Moderate | Severe |
| inflammation | | Present in fewer | Present in more | Present in majority |
| | | than 30% of portal | 30%-50% of portal | (more than 50%) |
| | | tracts | tracts | of portal tracts |
| Lobular | Absent | Mild | Moderate | Severe |
| Inflammation | | With little hepatocytes | With focal necrosis | With |
| | | necrosis or apoptosis | Causing confluence | bridging/severe |
| | | | and/or several | parenchymal |
| | | | apoptosis | necrosis |
| Bile duct | Absent | Mild | Moderate | Severe |
| injury | | Slight duct epithelium | Duet epithelial | Extensive duct |
| | | disorder with cytoplasmic | disorganization with partial | epithelial |
| | | eosinophilia | necrosis of affected ducts | disorganization |
| | | | | with complete |
| | | | | necrosis of |
| | | | | affected ducts |
| Bile duct loss | Absent | Mild | Moderate | Severe |
| | | Affecting 30% or | Affecting 30-60% | Affecting more |
| | | less | | than 60% |
| Cholestasis | Absent | Mild | Moderate | Severe |
| | | Visible only at 20X or | Visible at 5-10x | Visible easily at 5x |
| | | higher magnification | magnification but | magnification |
| | | | not easily | |
| Total Score | | | | |

Lung GVHD Scoring

| | | | | |
|---|---|---|---|---|
| inflammation | Absent | Mild | Moderate | Severe |
| | | Present around | Present with | Present with |
| | | vessels without | endotheliitis +−/ | extensive |
| | | endotheliitis | septal | septal |
| | | | expansion +/− mild | expansion, |
| | | | alveolar extension | heavy |
| | | | | intra-alveolar |
| | | | | inflammation |
| | | | | +/− |
| Bronchial/bronchiolar | Absent | Mild | Moderate | Severe |
| Epithelial injury | | Subepithelial | Subepithelial inflammation with | Subepithelial |
| | | inflammation with no | intra-epithelial inflammation but | inflammation |
| | | intra-epithelial | no epithelial necrosis and/or | with |
| | | inflammation | apoptosis | intra-epithelial |
| | | | | inflammation |
| Total Score (/6) | | | | |

### Results

### Ex Vivo Expansion and Characterization of Clinical-Grade DNTs

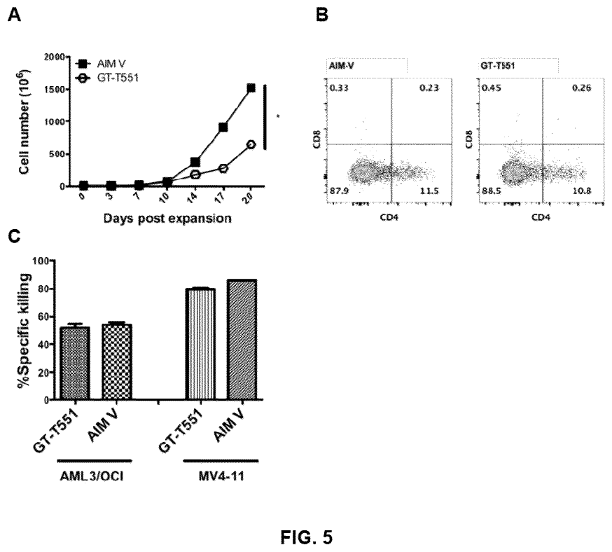

Expansion of a cellular product under GMP conditions is required before its clinic application. Our previous studies were conducted with DNTs expanded using research grade reagents containing xenogeneic components.^{14,15} Use of animal-derived supplements restrict the use of final products for patient treatment due to risks associated with the xenogeneic additives.²⁴ To expand DNTs under GMP conditions, clinical-grade reagents were used and the yield, purity, and function of DNTs expanded using different types of animal-serum free media were compared (FIG. 5). Subsequently, several parameters were investigated including cell concentration, IL-2 concentration, and the schedule of cell splitting. With an improved expansion method described herein, DNTs were expanded from 11 donors in 13 cultures. By 17 days of expansion, 1.11±0.63×10⁸ DNTs were generated from each milliliter of peripheral blood (PB; FIG. 1A) with an average fold expansion of 1558±795.5 (FIG. 1B) and an average purity of 91.9%±4.29% (FIG. 1C). To confirm our methods, three “validation-runs” were conducted in GMP-certified facilities. Similar yield, purity and cell stability in the infusion buffer were seen in all three runs, and all 3 batches of expanded DNTs passed tests for sterility, mycoplasma, and endotoxin.

To characterize the expanded DNTs, cell surface molecule expression profiling was performed on ex vivo expanded DNTs from three donors using the high-throughput flow cytometry screening method developed by Gedye C., et al.²³ DNTs stained with antibodies against 385 different surface molecules were analyzed by flow cytometry. The validity of the screening method was confirmed by checking the expression of T cell associated markers, CD2, CD3, and CD5, and lack of expression for B cell markers, CD19 and CD20 (FIG. 1D). Overall, ex vivo expanded DNTs display an effector memory T cell phenotype with expression of CD45RA, CD44, CD43, and CD49d and low or lack of CCR7, CD62L, BTLA, and CD127 expression (FIG. 1E). Ex vivo expanded DNTs were positive for chemokine receptors, CXCR3 (38.0%), CCR3 (42.6%), CCR6 (20.1%), and CCR9 (17.6%) (FIG. 1F), all of which are associated with recruitment of immune cells to inflammatory regions²⁵⁻²⁷, but were negative for other chemokine receptors.

To better understand immune responses mediated by DNTs, expression of cytotoxic molecules (FIG. 1G), costimulatory molecules, (FIG. 1H) and coinhibitory molecules (FIG. 1I) were examined. Amongst the cytotoxic molecules, two previously identified molecules involved in DNT-mediated anti-leukemia activity, NKG2D and DNAM-1 were expressed at high levels (83.3% and 77.3%, respectively), Lower levels of other cytotoxic molecules, NKp30 (13.4%), KIR2DS4 (15.2%), and membrane-bound TRAIL (16.3%) were detected, but DNTs were negative for FasL, NKp44, NKp46, and KIR3DS1. DNTs expressed costimulatory molecules CD30 (49.5%), GITR (22.5%), CD27 (15.3%) and CD28 (25.2%), but expression of OX40, CD40, 4-1BB, and HVEM was very low or absent. Unlike most ex vivo expanded effector T cells, expanded DNTs were low for coinhibitory molecules, ICOS, CTLA-4 and PD-1, and PD-1 ligands, suggesting that DNTs may be resilient to T cell exhaustion or cancer immune escape mechanisms. However, high expression of TIM-3 (65.7%), LAIR1 (95%), and NKG2A/CD94 (58.9% and 42.6%) were also detected, suggesting a potential inhibitory activity of these molecules on DNT-mediated anti-cancer activity.

Combining the use of anti-TIM3, anti-NKG2A, and anti-CD94 antagonistic antibodies and anti-CD27, anti-CD28, anti-GITR, or anti-CD30 agonistic antibodies with DNT may promote the activities of DNTs. As migration of DNTs to target tissue is required for its activity, chemokine receptor expression pattern on DNTs may be used to promote migration of DNTs to desired tissues.

As shown in FIGS. 1J and 1K, addition of different antibodies can modulate the cytotoxicity of DNTs against AML. For example, addition of TIM-3 antibody reduced the level of killing mediated by DNTs against a relatively more resistant cell line, AML3/OCI, while cytotoxicity against highly susceptible leukemia line, MV4-11, remained comparable. In contrast, addition of anti-CD3 antibody increased the cytotoxicity of DNTs against AML3/OCI

### Expanded DNTs Target Various Types of Cancers In Vitro and In Vivo in a Non-Donor Restricted Manner

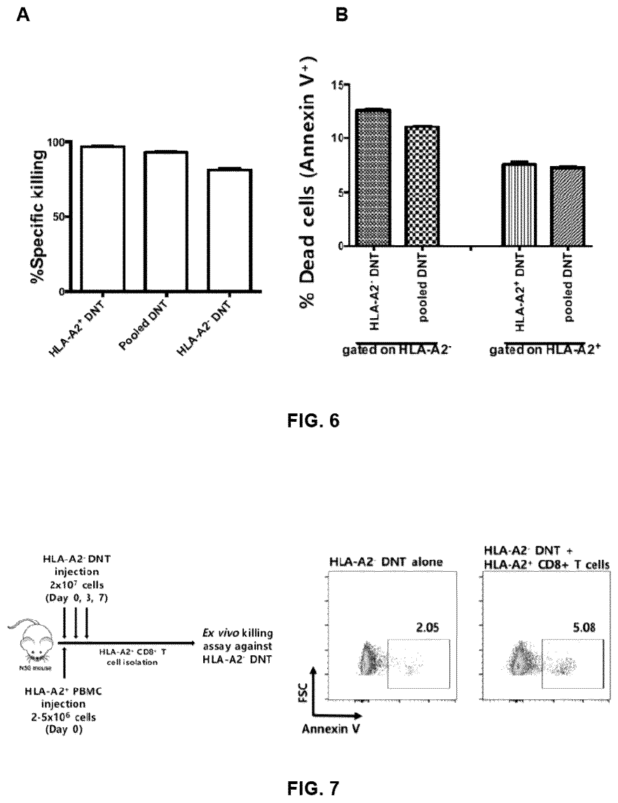

For an off-the-shelf ACT, cells manufactured from a single donor should be able to target cancers from multiple patients in a donor-unrestricted manner. To determine the function of clinical-grade DNTs, the cytotoxicity of expanded cells towards various cancer cell lines derived from myeloma, T cell leukemia, Burkitt's lymphoma, AML, EBV-LCL, large cell lung cancer, and lung adenocarcinoma was examined in vitro. DNTs exhibited broad anti-cancer cytotoxicity toward all of the cancer targets tested (FIG. 2A). Furthermore, clinical-grade DNTs from a single donor effectively targeted multiple cancer targets, OCI/AML3 and MV4-11 and a primary AML sample (FIG. 2B) and similar levels of cytotoxicity were mediated by DNTs from six different donors against the same cancer target (FIG. 2C). Further, mixing DNTs expanded from two different donors did not compromise the overall cytotoxicity compared to that induced by DNTs from either donor alone and donor DNTs showed no alloreactivity against each other when mixed in vitro (FIG. 6). These results collectively demonstrate that DNTs induce a broad cancer-specific cytotoxicity in a donor-unrestricted fashion.

To determine the anti-tumor activity of DNTs in vivo, xenograft models were established with human EBV-LCL and AML in immunodeficient NOD.Cg-Prkdcscid Il2rg^{Im1Wji}/SzJ (NSG) mice. In EBV-LCL xenograft model, mice inoculated with a lethal dose of EBV-LCL were treated with PBS or DNTs. All of the mice in the PBS-treated group died within 28 days (n=6), whereas four out of six DNT-treated mice survived through the length (85 days) of this study (FIG. 2D). Similarly, mice bearing human AML cells treated with DNTs showed 17.1-fold reduction in AML engraftment level in the bone marrow (2.37±0.749%) compared to those treated with PBS (40.5±4.56%) (FIG. 2E). Interestingly, DNTs displayed a potent cytotoxic activity against primary AML patient cells expressing markers associated with leukemic blasts (CD33⁺ CD45^{kw} CD34⁺ and CD33high CD34⁺) but not those with phenotype associated with normal cells (CD33⁻ CD45^{high} CD34−) (FIG. 2F) in the same killing assays, indicating that DNTs preferentially target leukemic cells but not normal cells. To further validate this result, the level of tissue damage caused by DNTs was assessed in an AML-xenograft model as described in 2E, and compared with those caused by PBMC. Consistent with the in vitro findings, a significant anti-cancer activity of DNTs was observed in xenograft models, but none of the cancer-bearing DNT-treated mice exhibited signs of xenogeneic GvHD (FIGS. 2G and 2H). Liver tissue from PBMC-treated mice showed moderate portal lymphocyte infiltration and severe bile duct injury, whereas. DNT-treated mice showed mild portal lymphocyte infiltration without bile duct, vascular or other injuries (FIG. 2G). In lungs, PBMC-treated mice showed severe inflammation around vessels and bronchioles, endothellitis, and septal inflammation around alveoli. In contrast, DNT-treated mice showed no inflammation around vessels and bronchioles, and no endotheliitis or septal inflammation around alveoli were seen. The tissue damage in the livers and lungs was blindly scored by a pathologist and the DNT-treated group scored significantly lower than that from the PBMC-treated group (FIG. 2H). Taken together, these data demonstrate that HD-derived DNTs expanded under GMP conditions are effective at targeting a broad range of cancer types in vitro and in xenograft models in a donor-unrestricted fashion without off-tumor toxicity, which are necessary features for a successful off-the-shelf allogeneic ACT.

### Expanded DNTs can be Cryopreserved Under GMP Conditions

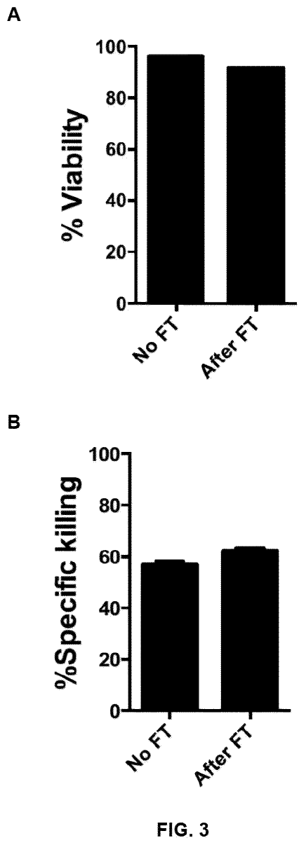

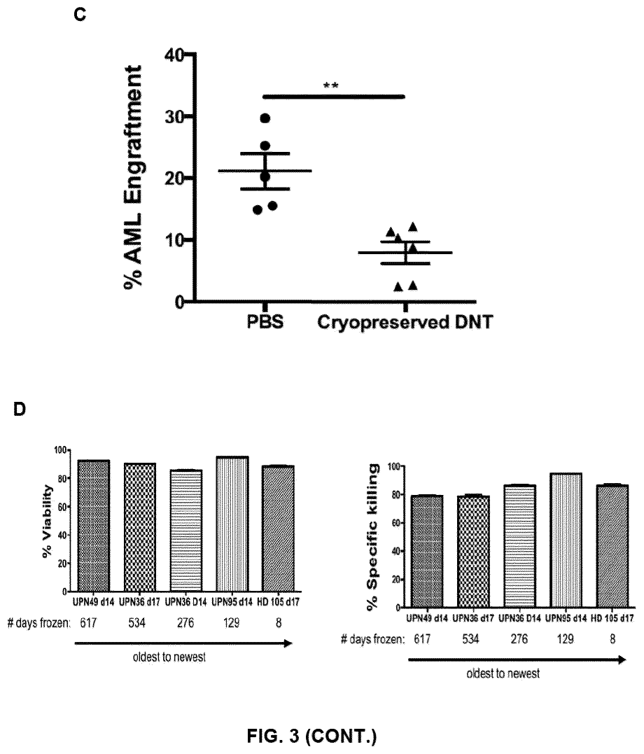

Often the importance of cryopreserving expanded cells under a clinically compliant condition with negligible effect on cell viability and function is overlooked.²⁸ Effective cryopreservation methods allow for storage of manufactured cells, increases the consistency of cell products, offers immediate availability of cell therapy for patient infusion, and provides a way to distribute cell therapy to areas that cannot manufacture cells, altogether increasing the flexibility and accessibility of ACT. To this end, multiple parameters were investigated to cryopreserve ex vivo expanded DNTs in a GMP-compliant animal serum-free media, including DMSO concentration, methods to add and remove DMSO, re-stimulation of thawed DNTs, and cell concentration for freezing. Optimally cryopreserved DNTs retained their viability (FIG. 3A) and anti-leukemic activity in vitro (FIG. 3B) and in a xenograft model (FIG. 3C). To use cryopreserved DNTs as a ready-to-go product, their shelf-life as frozen cells is important. To investigate this, the viability and cytotoxicity of DNTs frozen for 617, 534, 276, 129, or 8 days were determined. All DNTs remained viable and retained their anti-leukemic function (FIG. 3D). Collectively, DNTs expanded under clinically acceptable conditions can be cryopreserved in GMP-compliant media for at least 600 days without compromising their function, providing a way to use allogeneic DNTs as a “ready-to-go” treatment for cancer patients.

### Live DNTs Do Not Activate Allogeneic Immune Response In Vitro and Can Persist In Vivo

The ability of adoptively transferred cells to persist in recipients affects the outcome of ACT²⁹, and its importance is more apparent in allogeneic settings as Infused cells can be recognized and rejected by a patient's immune system³⁰. Thus, the persistence, proliferative capacity, and migration patterns of DNTs in vivo were determined by systemically injecting CFSE-labeled ex vivo expanded human DNTs into naïve sublethally irradiated NSG mice. Cells from peripheral blood, spleen, bone marrow (BM), liver and lung were obtained on days 2, 7, 10 and 14 post-infusion. DNTs were detected in all tissues examined two days after infusion, and the frequency of DNTs decreased thereafter, but cells were still detectable 14 days after injection (FIG. 4A). Compared to day 0, CFSE fluorescence intensity was diluted up to day 10 post-infusion but no further reduction from day 10 to day 14 (FIGS. 4B and 4C). These results demonstrate that DNTs migrate to different tissues and undergo limited cell division in vivo and can be detected for at least two weeks after infusion into naive NSG mice.

The persistence of DNTs in naïve NSG mice, however, does not reflect their persistence in a clinical setting as immunodeficient mice are not able to reject the infused cells. To determine whether DNTs are prone to rejection by allogeneic immune responses, we set up classical mixed-lymphocyte reaction (MLR) assays as shown schematically in FIG. 4D. CFSE labeled PBMCs from one donor (HD1) were stimulated with live or irradiated ex vivo expanded DNTs from the same donor (HD1) or a different donor (HD2) to determine the immunogenicity of allogeneic DNTs to conventional T cells, Stimulation with irradiated allogeneic DNTs increased proliferation of CD4⁺ and CD8⁺ T cells by 27.7%±0.12% and 37.7%±0.91%, respectively, which was significantly higher than that by irradiated autologous DNTs (13.7%±0.94% for CD4⁺ and 10.8%±0.12% for CD8⁺ T cells; FIG. 4E). Interestingly, significantly lower degree of proliferation of CD4⁺ (6.04%±0.69%) and CD8⁺ T cells (6.32%±0.55%) was Induced by live allogeneic DNTs, which were comparable to the levels of proliferation induced by live autologous DNTs (6.61%±0.38% for CD4⁺ and 6.79%±0.21% for CD8⁺ T cells; FIG. 4E). To further determine whether CD8⁺ T cells co-cultured with autologous or allogeneic DNTs were able to target allogeneic DNTs, the CD8⁺ cells were isolated 4-6 days after coculture and used as effector cells against autologous or allogeneic DNTs as illustrated in FIG. 4D. As expected, CD8⁺ T cells stimulated with live or irradiated autologous DNTs did not Induce any cytotoxicity to allogeneic DNTs. Importantly, whereas CD8⁺ T cells stimulated by irradiated allogeneic DNTs elicited potent cytotoxicity against allogeneic DNTs in a dose-dependent manner, no cytotoxicity of CD8⁺ T cells against allogeneic DNTs was detected when the CD8 cells were stimulated with live DNTs (FIG. 4F). Collectively, these results demonstrate that while DNTs carry alloantigens, live allogeneic DNTs do not induce significant levels of alloreactivity of conventional T cells in vitro.

To further confirm that viable DNTs are resistant to allogeneic immune response in vivo, naïve NSG mice were infused with PBMC from an HLA-A2⁺ donor followed by 0 or 3 injections of HLA-A2⁻ DNTs from another donor. Four-weeks post-infusion, cells from the spleen, bone marrow, and lungs were isolated and engraftment of human T cells was determined. We found that in 4 out of 5 treated mice, a significant level of HLA-A2⁻ DNTs co-engrafted with HLA-A2⁺ CD4⁺ and CD8⁺ T cells in the same tissues (FIG. 4G), indicating that DNTs persisted in the recipients for at least 4 weeks in the presence of allogeneic conventional T cells. To further investigate the allo-reactivity of engrafted CD8⁺ T cells, HLA-A2⁺ CD8⁺ T cells were subsequently isolated from DNT-and PBMC-treated mice and used as effectors against DNTs from the same donor origin as used for the xenograft experiment. No significant decrease in DNT cell viability was seen in the presence of isolated HLA-A2⁺ CD8⁺ T cells (FIG. 7), further supporting that allogeneic CD8⁺ T cells did not cause elimination of DNTs in a xenograft model. Collectively, these data suggest that ex vivo expanded DNTs are resistant to allogeneic immune cell-mediated rejection and warrant further testing the potential of allogeneic DNTs as an off-the-shelf ACT that is resistant to HvG rejection in a clinical setting.

### Discussion

While the efficacy and safety of a novel therapy are the priorities in the development of a new treatment, feasibility of translation into patient treatment should not be overlooked. Current forms of ACT take the “personalized” approach, generating cellular product for individual patients “on-demand” using a patients' own Immune cells and have achieved remarkable results in some patients but factors limiting their broad application have also become apparent from these studies.^{31,32} Therefore, off-the-shelf cell therapy that will allow more patient-accessibility is considered as the next generation of ACT.^{12,33}

An essential property of an off-the-shelf cellular product is its ability to target a broad range of cancers in a donor-independent manner. We have demonstrated that clinical-grade DNTs target an array of hematological and solid cancers in vitro (FIG. 2A) and EBV-LCL and AML in xenograft models (FIGS. 2D and 2E, respectively). In addition to cancer types presented in this study, we observed significantly inhibited non-small cell lung cancer progression after DNT treatment in xenograft models.³⁴ Notably, DNTs from a single donor could kill cancer cells of different origins (FIG. 2B), and the level of cytotoxicity against the same cancer target was comparable between DNTs derived from different donors (FIG. 2C) without observed off-tumor toxicity (FIGS. 2F and 2G). Further, cryopreservation of expanded DNTs under clinically compliant conditions did not compromise the function of DNTs (FIG. 3). Collectively, these findings highlight the possibility of cryopreserving a large batch of expanded DNTs as a ready-to-go treatment for multiple patients.

One of the major concerns of using allogeneic T cells as a cellular therapy is the risk of GvHD.⁶ Jacoby et al. reported that infusion of allogeneic CD19-CAR T cells induced severe GvHD in the recipient mice due to the recognition of host allo-antigens by the endogenous TCR.³⁵ To address this issue, several groups knocked out the endogenous TCR from CD19-CAR T cells¹⁸ or transgenic TCR transduced T cells³⁶ and successfully prevented the onset of GvHD and maintained the anti-cancer activity. However, such approaches are limited to ACT against known tumor antigens as the use of exogenously transduced receptors against cancer-associated antigens is required to replace the lost anti-tumor specificity of the endogenous TCR, and impose another hurdle in cell production. We and others have previously shown that Infusion of allogeneic mouse DNTs or xenogeneic human DNTs does not cause GVHD, unlike conventional CD4⁺ and CD8⁺ T cells.^{14,37} In line with previous findings, DNTs specifically targeted leukemic cells while sparing normal cells from the same patient sample in the same cytotoxicity assay (FIG. 2F), and cancer-bearing mice treated with expanded human DNTs showed significant reduction in leukemia load without signs of toxicities on normal mouse tissues (FIG. 2G). Taken together, these studies demonstrate that infusion of non-genetically modified allogeneic DNTs is unlikely to cause GvHD.

The persistence of infused immune cells has been shown to be correlated with treatment outcomes.¹⁹ Persistence of infused T cells are decided by intrinsic and extrinsic factors. As a cell intrinsic factor, the activation status of infused cells can affect their persistence. We found that when DNTs were injected alone, they migrated and persisted in various tissues including the liver, lung, blood, bone marrow, and spleen of NSG mice up to 14 days (FIG. 4A). Based on surface molecule profiling data, DNTs exhibited an effector memory phenotype (FIG. 1E) that is associated with a more robust immune response and shorter persistence compared to central memory T cells.¹⁹ While the persistence of infused allogeneic DNTs in patients is currently under study in our phase I clinical trial, modifications to the cell expansion method may help to generate DNTs with a central memory phenotype if longer persistence is desired. Alternatively, such an issue can be overcome by using the off-the-shelf property of DNTs and re-infusing patients with cryopreserved DNTs as needed.

The major extrinsic factor affecting cell persistence is the rejection of infused cells by the host-immune system. Many studies on allogeneic ACT focuses on avoiding GvHD, but less so on HvG rejection. In one study conducted by Torakal et al., HLA expression on allogeneic CAR-T cells was knocked out by genetic manipulation, which successfully evaded allogeneic CD8⁺ T cell-mediated cytotoxicity²¹. However, the feasibility and safety of this approach in a clinical setting need to be validated. Interestingly, we found that stimulation of conventional T cells with live allogeneic DNTs does not elicit cytotoxic toward the allogeneic DNTs (FIG. 4E). Further, co-infusion of allogeneic DNTs with PBMCs from a different donor resulted in their co-engraftment in NSG mice (FIG. 4G) and co-engrafted allogeneic CD8⁺ T cells showed no allo-reactivity against DNTs (FIG. 7). To our knowledge, this is the first form of allogeneic ACT that is resistant to HvG reaction without genetic modification or exogenous immunosuppressant. Nevertheless, given that irradiated allogeneic DNTs were potent stimulators of allo-reactive T cells, DNT viability for patient infusion needs to be carefully monitored to prevent. the onset of HvG reaction.

The mechanisms by which live allogeneic DNTs evade rejection by conventional T cells are under study. We have shown previously that mouse DNTs could kill allo-reactive CD8⁺ T cells that were activated by the same alloantigens that activated DNTs.³⁸ Furthermore, a higher DNTs versus CD8⁺ T cell ratio correlates with a less severe GvHD in allogeneic hematopoietic stem cell transplantation patients.³⁹ Collectively, these data support that, unlike conventional T cells, infusion of viable allogeneic DNTs is less likely to result in rejection as the result of HvG reaction.

NK cells have potential to be used as an off-the-shelf therapy without genetic modification due to HLA-unrestricted anti-tumor function and limited GvHD causing activities.⁴⁰ NK-92, a cell line derived from a patient NK cell lymphoma, has been shown to be safe and feasible as an off-the-shelf ACT in clinical studies.⁴⁰ However, only one study reported that out of 15 treated patients, two had mixed responses and one had stable disease. Limited anti-tumor activity may be due to short persistence as NK-92 were detectable only for ˜48 hours after infusion⁴¹, possibly due to irradiation of the cells prior to patient infusion to avoid potential in vivo tumorigenesis as they are immortalized cells. It is also possible that the infused NK92 were rejected due to HvG rejection⁴¹, which has not yet been studied. In a clinical study conducted by Romee et al., infusion of cytokine-induced memory like allogeneic primary NK cells showed a more promising clinical response, where four out of nine AML patients achieved complete remission, in the absence of dose limiting toxicity.⁴² However, donor-derived NK cells were not detectable by two to three weeks post infusion, suggesting that the host-immune system recovered and rejected donor-derived allogeneic NK cells or infused NK cells have a limited life expectancy. We found that significant numbers of DNTs were detected in mice 4 weeks after co-infusion with allogeneic PBMCs in the absence of lymphodepletion, suggesting a better in vivo persistence of allogeneic DNTs than NK cells (FIG. 4G).

In our previous study¹⁴, we have directly compared the cytotoxic activity of DNTs with NK-92 and expanded primary NK cells. We showed that while similar toxicity was seen against a known NK cell target, K562, superior killing was mediated by DNTs against all seven AML targets tested, including four samples resistant to NK-92 mediated cytotoxicity. Similarly, DNTs derived from healthy donors showed superior killing against AML cell lines than those of primary activated NK cells. This suggests that DNTs function through a different mechanism than that of NK cells, and DNTs may be able to target cancers resistant to NK-92. Further, resistance of DNTs to HvG reaction may result in a longer persistence of DNTs in patients and therefore a more prolonged effect.

In summary, we have established a method to expand clinical-grade DNTs from healthy donors without genetic modification or extensive manipulations. To our knowledge, DNTs are the first T cell ACT that fulfills all the requirements of an off-the-shelf allogeneic cell therapy without genetic alteration. The expanded DNTs can be cryopreserved, persist in an allogeneic environment in the absence of immunosuppression and are effective in targeting various cancers without off-tumor toxicity. These properties allow for the use of allogenelc DNTs as an off-the-shelf ACT for patients with different cancer types as a stand-alone therapy or in combination with other conventional therapies. Further, DNTs can also be used in combination of antibodies that can modulate such as Tim-3, CD94/NKG2A, LAIR-1, CCR3, and CXCR3.

### Example 2: Cryopreservation and Off-the-Shelf Potential of Allogenic DNTs

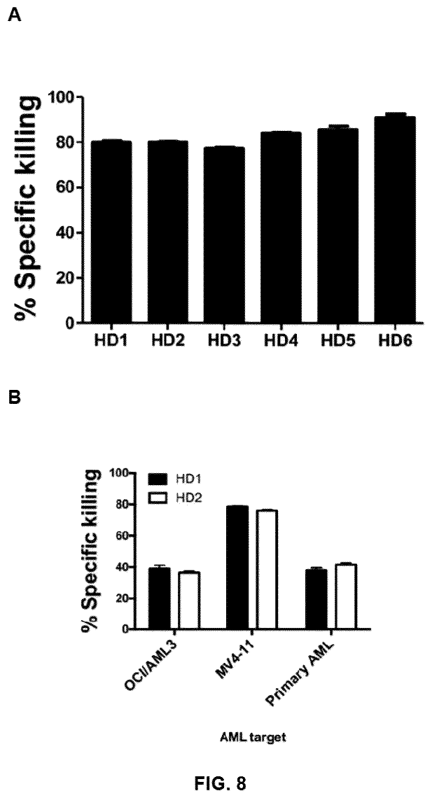

It has been determined that allogeneic DNTs expanded from different healthy donors showed similar level of cytotoxicity against a same AML target (FIG. 8), and DNT cells from a single donor could target AML cells obtained from an array of AML patients (FIG. 8), without any observed toxicity against normal cells. These findings suggest that the level of response by DNT therapy will be comparable regardless of the donor of which DNTs are derived from, and that DNTs from a single donor can be used to treat multiple AML patients, supporting the potential for utilizing allogeneic DNT cells as an “off-the-shelf” therapeutic approach. However, lack of a storage method that can preserve the anti-leukemic function of ex vivo expanded DNTs impedes their use as “off-the-shelf” therapeutics.

### Functional Analysis of Expanded Cryopreserved DNTs

To determine the anti-leukemic function, cryopreserved DNTs were used as effector cells in in vitro cytotoxicity assay against cancer cell lines and none-frozen DNTs from the same donor or culture was used as a control. The anti-leukemic function of cryopreserved DNTs were further validated in an AML xenograft model.

### Cryopreservation of Ex Vivo Expanded DNTs.

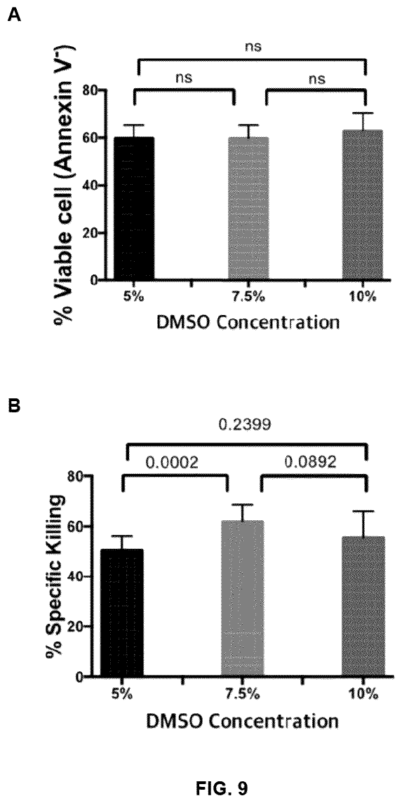

While the applicants have established a standard protocol to cryopreserve non-expanded DNT cells¹⁵, the same protocol could not preserve the function and viability of ex vivo expanded DNTs. Several parameters were modified to develop an optimal freezing method for ex-vivo DNTs. First, the optimal concentration of DMSO in freezing media and method of adding DMSO was determined by comparing the viability and cytotoxic function of DNTs frozen in FBS+5% DMSO, FBS+7.5% DMSO, and FBS+10% DMSO, It was found that DNTs frozen in FBS+7.5% had higher viability and cytotoxic function than those cells frozen in FBS+5% DMSO or 10% DMSO (FIG. 9)

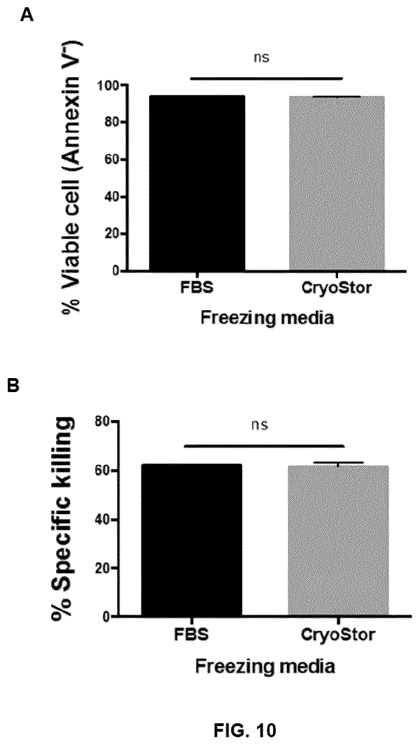

To test whether the use of animal serum can be avoided in the freezing process to ease the transition of the technology into clinic, the viability and cytotoxic function of DNTs frozen in FBS+7.5% DMSO and Cryostor+7.5% DMSO were compared, and showed that the viability (FIG. 10) and cytotoxic function (FIG. 10) of cells from both freezing reagent were comparable.

### Validating the Function of Ex Vivo Expanded DNTs Post-Cryopreservation.

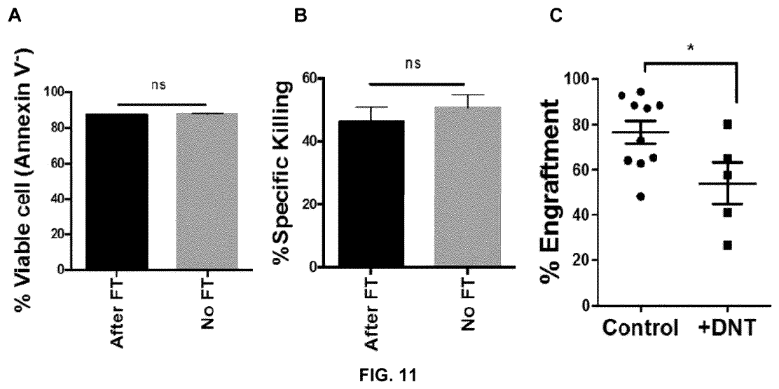

While above studies showed the optimal method to cryopreserve expanded DNTs, we wanted to ensure that cryopreserved DNTs do not have compromised function compared to non-frozen DNTs. To this end, the in vitro anti-leukemic function expanded cryopreserved DNTs using the optimized protocols described herein was compared to non-frozen DNTs expanded from the same donor or the same expansion culture. As showed in FIG. 11 that both the viability and the cytotoxic function of DNTs were comparable. To further confirm that in vivo function of frozen cells is preserved, immunodeficient NSG mice were engrafted with primary AML blasts were treated with thawed DNTs. Similar to none-frozen DNTs, cryopreserved DNTs significantly reduce the level of AML engraftment in a xenograft model (FIG. 11).

Taken together, we have developed a method for cryopreservation of ex vivo expanded DNTs by optimizing each step of cryopreservation methods. Using this method, we demonstrate that ex vivo expanded DNTs can be cryopreserved and maintain their anti-tumor function both in vitro and in vivo. This method allows DNTs to be stored as an “off-the-shelf” living drug to treat patients in need. It may also be applied to cryopreserving other expanded anti-tumor T cells, NKT cells and NK cells for their research and clinical use,

### Example 3: Expansion of Clinical Grade DNTs for Off-the-Shelf Allogenic Cell Therapy

Patent application no. PCT/CA2006/001870 describes a method for ex vivo expansion of double negative T (DNT) cells. Using that method it is possible to generate 2.5×10⁶ DNT cells from one milliliter of blood using expansion methods and reagents involving xenogeneic-additives. However, to produce DNTs for use in an off-the-shelf therapy, where DNTs obtained from a single expansion can be used for multiple treatments and/or patients, a higher DNT-yield is needed. Further DNTs generated using the previous method were research-grade. To allow translation of DNT therapy to clinic, establishing methods to 1) improve the final cell yield 2) using clinically-compliant expansion methods and reagents were needed. Here, a new ex vivo DNT cell expansion protocol is described that results in clinical-grade DNTs with significantly improved the yield at the end of expansion.

Various parameters, such as cell concentration during expansion, days of cell splitting, type and concentration of additives given during expansion, and different types of clinical-grade culture medium, were investigated. DNTs from healthy donors (HDs) expanded using newly established GMP-grade expansion method result in significantly higher number of DNTs at the end of expansion compared to those expanded using previously defined research-grade expansion method (FIG. 12).

As shown in Example 1 and FIG. 5, DNTs expanded using two different clinically compliant culture media (AIM V and GT-T551) resulted in a significant difference in expansion. AIM V produced a higher number of cells and was used for subsequent DNT expansions.

The addition of plasma (FIG. 13A), but not human serum albumin (HSA; FIG. 13B), also significantly improves the expansion of HD-DNTs ex vivo.

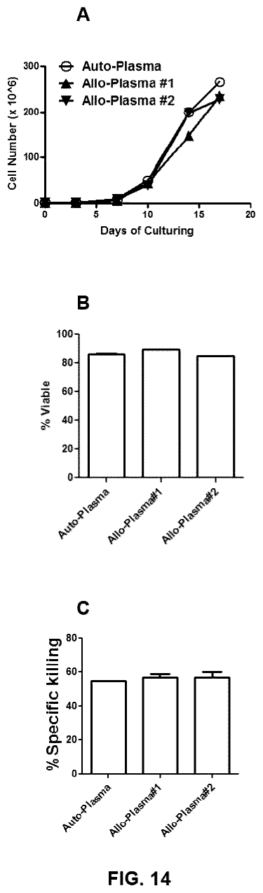

Surprisingly, DNTs can be expanded using plasma from allogeneic sources which give comparable expansion profile (FIG. 14A), viability (FIG. 14B), and cytotoxicity against cancer cells (FIG. 14C) as using autologous plasma.

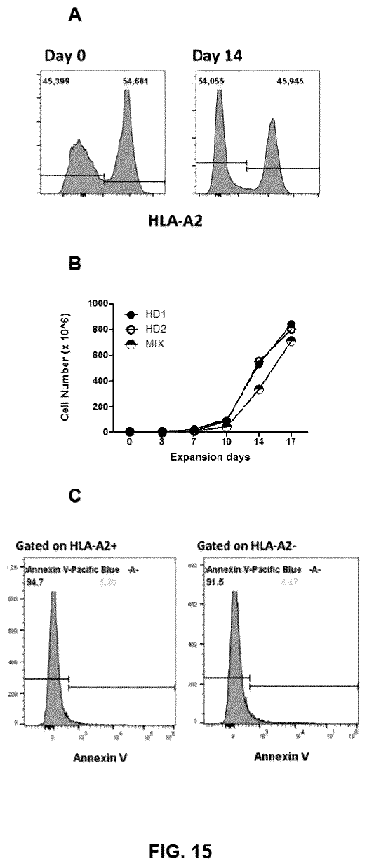

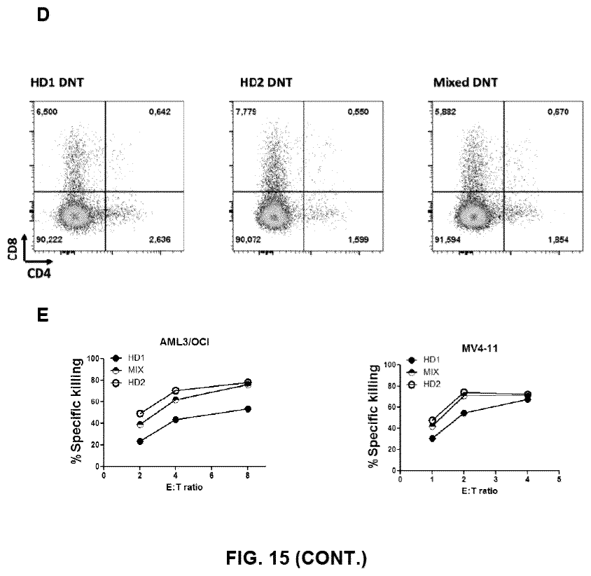

Surprisingly, DNTs derived from different donors can be mixed and expanded in the same culture (FIG. 15A) without hampering their expansion profile (FIG. 15B), viability (FIG. 15C), purity (FIG. 15D) or anti-cancer activity (FIG. 15E).

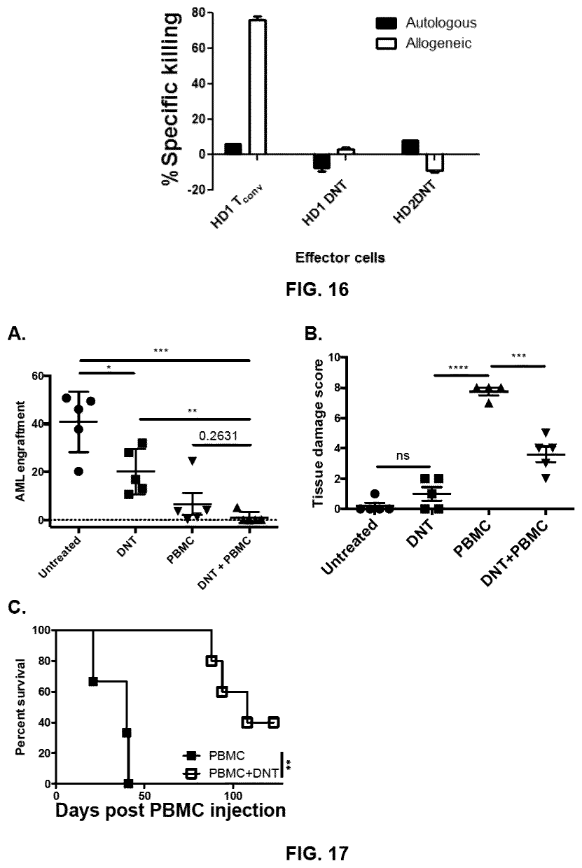

To determine if DNTs expanded from pooled donor develop alloreactivity to each other during expansion, different donor derived DNTs were Isolated using differentially expressed HLA subtypes and their cytotoxicity tested against each other. As shown in FIG. 16, while conventional T cells derived from the same donors develop potent allo-reactivity, DNTs cocultured for 17 days do not development of allo-reactivity against each other.

Despite allo-HSCT being the only consolidation therapy for many of hematological malignancies with long-term curative potential, its efficacy and safety need to be improved. To determine if DNTs can be used as an adjuvant to improve the efficacy and safety of allo-HSCT, cancer-bearing mice were infused with PBMC with or without DNTs. Notably, co-infusion of DNTs with PBMC showed superior anti-leukemic activity than that of DNTs alone, and completely eradicated the cancer in all five treated mice, as oppose to 1 out of five treated mice with detectable level of cancer in PBMC-alone group (FIG. 17A). Importantly, DNT co-infusion reduced the degree of GVHD induced by PBMC-derived T cells (FIG. 17B). This was further validated in a GvHD-xenograft model, where DNT-infusion significantly prolonged survival of mice treated with PBMC (FIG. 17C).

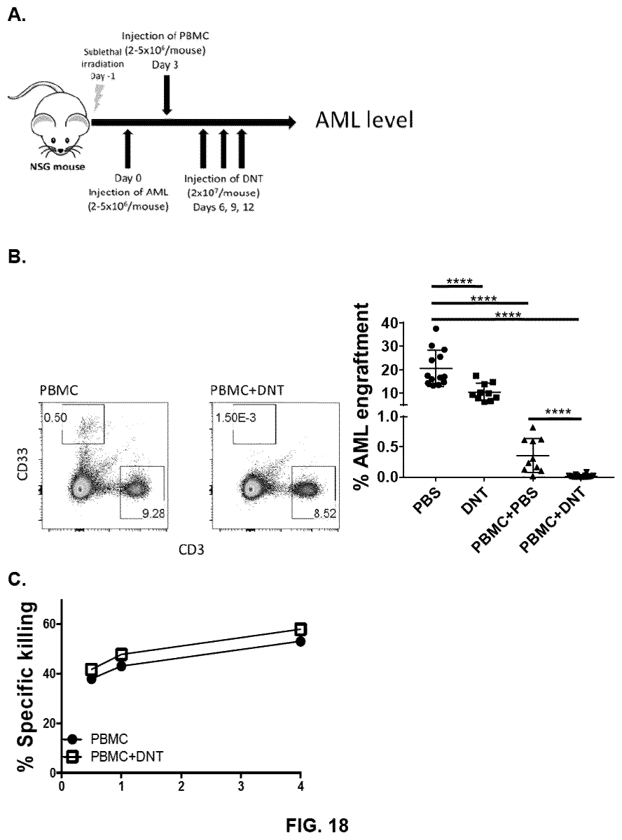

Next, to determine if DNTs can enhance the overall anti-leukemia effect in leukemia-bearing host treated with PBMCs, NSG mice engrafted with an aggressive human AML cell line MV411 were treated with PBS, human PBMCs, ex vivo expanded DNTs or human PBMC followed by DNTs, and the leukemia engraftment level in the bone marrow was assessed (FIG. 18A). As previously reported¹⁶, treatment with DNTs resulted in 50% reduction (from 20.6%±7.8% to 10.5%±3.7%) in leukemia burden compared to PBS control group, but the effect was incomplete (FIG. 18B). PBMC-derived T_{conv} cells mediated a strong anti-leukemia response, reducing the AML level to 0.68%±0.27%, yet there were detectable residual leukemic cells in the bone marrow (FIG. 18B). Interestingly, AML cells were not detectable in bone marrows of mice treated with PBMC followed by DNTs (FIG. 18B), Given that CD8⁺ T cells are involved in both GvL and GvHD in PBMC treated group and that DNTs attenuate the severity of GVHD, the effect of DNT co-treatment on CDB T cell-mediated GvL activity in PBMC+DNT-treated group was compared to that of PBMC-treated group. CD8⁺ T cells were isolated from PBMC− and PBMC+DNT-treated mice and used as effector cells against the leukemic cells initially used for the xenograft experiment. It was found that CD8⁺ T cells from both groups induce significant and comparable degree of cytotoxicity against the AML cells ex vivo (FIG. 18C), suggesting that DNTs do not negatively affect the anti-leukemic activity of T_{conv} cells, while inducing anti-leukemia activity of their own to yield in a greater anti-leukemia activity in xenograft models. Together, these data support the notion that DNTs do not dampen GvL effect of T_{conv} cells, rather, they can increase the overall anti-leukemic response, which may lead to eradication of the disease.

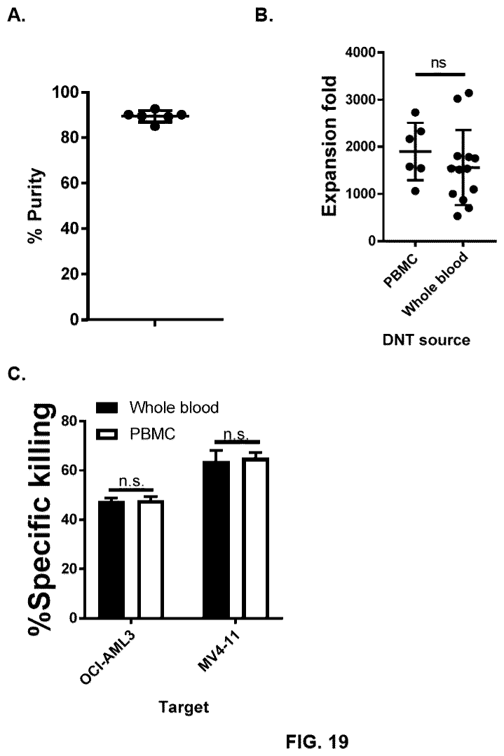

Currently used methods of DNT isolation require obtaining whole blood from donors as CD4+ and CD8+ cells are depleted by cross-linking to erythrocytes. While this method has been successfully provided enough number to treat one to two patients, larger scale expansion is limited by the amount of blood volume that can be safely drawn from a donor. Therefore, a method to scale-up DNT expansion by utilizing leukapheresis sample, instead of whole blood, will increase the amount of DNTs that can be expanded at once and maximize the off-the-shelf potential of DNT therapy. To this end, a starting DNT cell population was isolated from PBMCs derived from leukapheresis using a selection column and DNTs with a mean purity of 89.5%±2.51% (FIG. 19A) were obtained. Further, DNTs obtained from PBMCs showed comparable expansion fold as those obtained from whole blood with an average expansion fold of 1899±615.7 (FIG. 19B). Lastly, DNTs expanded from PBMCs showed comparable in vitro cytotoxicity as those isolated and expanded from whole blood (FIG. 19C). These data demonstrate that DNTs can be isolated and expanded from PBMCs using the optimized method and show comparable characteristics as those isolated from whole blood.

Collectively, the results outlined in this patent demonstrate that DNTs can be isolated and pooled from multi-donor leukapheresis samples to allow for large scale expansions that can be used to manufacture enough cellular products treat hundreds of patients from a single expansion culture rather than 2-3 patients using a maximal amount of whole blood that can be obtained from a single donor at one time.

All publications, patents and patent applications cited herein are herein incorporated by reference in their entirety to the same extent as If each individual publication, patent or patent application was specifically and individually Indicated to be incorporated by reference in its entirety.

### REFERENCES

1 Park, J. H. et al. Long-Term Follow-up of CD19 CAR Therapy in Acute Lymphoblastic Leukemia. N. Engl. J. Med. 378, 449-459, doi:10.1056/NEJMoa1709919 (2018).
2 Tran, E. et al. T-Cell Transfer Therapy Targeting Mutant KRAS in Cancer. N. Engl. J. Med. 375, 2255-2262; doi:10.1056/NEJMoa1609279 (2016).
3 Kalos, M. & June, C. H. Adoptive T cell transfer for cancer immunotherapy in the era of synthetic biology. Immunity 39, 49-60, doi:10.1016/j.immuni.2013.07.002 (2013).
4. Maus, M. V. et al. Ex vivo expansion of polyclonal and antigen-specific cytotoxic T lymphocytes by artificial APCs expressing ligands for the T-cell receptor, CD28 and 4-1BB. Nat. Biotechnol. 20, 143-148, doi:10.1038/nbt0202-143 (2002).
5 FDA Approves Second CAR T-cell Therapy. Cancer Discov 8, 5-6, doi:10.1158/2159-8290.CD-NB2017-155 (2018).
6 June, C. H., Riddell, S. R. & Schumacher, T. N. Adoptive cellular therapy: a race to the finish line. Sci Transl Med 7, 280ps287, doi:10.1126/scitranslmed.aaa3643 (2015).
7 Dickinson, A. M. et al. Graft-versus-Leukemia Effect Following Hematopoietic Stem Cell Transplantation for Leukemia. Front Immunol 8, 496, doi:10.3389/fimmu.2017.00496 (2017).
8 Chang, X., Zang, X. & Xia, C. Q. New strategies of DLI in the management of relapse of hematological malignancies after allogeneic hematopoietic SCT. Bone Marrow Transplant. 51, 324-332, doi:10.1038/bmt.2015.288 (2016).
9 Devillier, R. et al. Outcome of relapse after allogeneic stem cell transplant in patients with acute myeloid leukemia. Leuk. Lymphoma 54, 1228-1234, doi:10.3109/10428194.2012.741230 (2013).
10 Zelser, R. & Blazar, B. R. Acute Graft-versus-Host Disease—Biologic Process, Prevention, and Therapy. N. Engl. J. Med. 377, 2167-2179, doi:10.1056/NEJMra1609337 (2017).
11 Zeiser, R. & Blazar, B. R. Pathophysiology of Chronic Graft-versus-Host Disease and Therapeutic Targets. N. Engl. J. Med. 377, 2565-2579, doi:10.1056/NEJMra1703472 (2017).
12 Ruella, M. & Kenderian, S. S. Next-Generation Chimeric Antigen Receptor T-Cell Therapy: Going off the Shelf. BioDrugs 31, 473-481, doi:10.1007/s40259-017-0247-0 (2017).
13 Chen, B., Lee, J. B., Kang, H., Minden, M. D. & Zhang, L. Targeting chemotherapy-resistant leukemia by combining DNT cellular therapy with conventional chemotherapy. J. Exp. Clin. Cancer Res. 37, 88, doi:10.1186/s13046-018-0756-9 (2018).
14 Lee, J. et al. Allogeneic human double negative T cells as a novel immunotherapy for acute myeloid leukemia and its underlying mechanisms. Clinical Cancer Research (provisionally accepted) (2017).
15 Merims, S. et al. Anti-leukemia effect of ex vivo expanded DNT cells from AML patients: a potential novel autologous T-cell adoptive immunotherapy. Leukemia 25; 1415-1422, doi:10.1038/leu.2011.99 (2011).
16 Lee, J. B. et al. Developing Allogeneic Double-Negative T Cells as a Novel Off-the-Shelf Adoptive Cellular Therapy for Cancer. Clin. Cancer Res. 25, 2241-2253, doi:10.1158/1078-0432.CCR-18-2291 (2019).
17 Legut, M., Dolton, G., Mian, A. A., Ottmann, O. G. & Sewell, A. K. CRISPR-mediated TCR replacement generates superior anticancer transgenic T. cells. Blood 131, 311-322, doi:10.1182/blood-2017-05-787598 (2018).
18 Poirot, L. et al. Multiplex Genome-Edited T-cell Manufacturing Platform for “Off-the-Shelf” Adoptive T-cell Immunotherapies. Cancer Res. 75, 3853-3864, doi:10.1158/0008-5472.CAN-14-3321 (2015).
19 Gattinoni, L., Klebanoff, C. A. & Restifo, N. P. Paths to stemness: building the ultimate antitumour T cell. Nat. Rev. Cancer 12, 671-684, doi:10.1038/nrc3322 (2012).
20 Torikai, H. & Cooper, L. J. Translational Implications for Off-the-shelf Immune Cells Expressing Chimeric Antigen Receptors. Mol. Ther. 24, 1178-1186, doi:10.1038/mt.2016.106 (2016).
21 Torikai, H. et al. Toward eliminating HLA class I expression to generate universal cells from allogeneic donors. Blood 122, 1341-1349, doi:10.1182/blood-2013-03-478255 (2013).
22 Rosenbaum, L. Tragedy, Perseverance, and Chance—The Story of CAR-T Therapy. N. Engl. J. Med. 377, 1313-1315, doi:10.1056/NEJMp1711886 (2017)
23 Gedye, C. A. et al. Cell surface profiling using high-throughput flow cytometry: a platform for biomarker discovery and analysis of cellular heterogeneity. PLoS ONE 9, e105602, doi:10.1371/journal.pone.0105602 (2014).
24 Chimenti, L et al. Serum and supplement optimization for EU GMP-compliance in cardiospheres cell culture. J. Cell. Mol. Med. 18, 624-634 doi:10.1111/jcmm. 12210 (2014).
25 O'Boyle, G. et al. Chemokine receptor CXCR3 agonist prevents human T-cell migration in a humanized model of arthritic inflammation. Proc. Natl. Acad. Sci. U.S.A. 109, 4598-4603, doi:10.1073/pnas.1118104109 (2012).
26 Hirota, K. et al. Preferential recruitment of CCR6-expressing Th17 cells to inflamed joints via CCL20 in rheumatoid arthritis and its animal model. J. Exp. Med. 204, 2803-2812, doi:10.1084/jem.20071397 (2007).
27 Oswald, C. et al. Intracellular allosteric antagonism of the CCR9 receptor. Nature 540, 462-465, doi:10.1038/nature20606 (2016).
28 Woods, E. J., Thirumala, S., Badhe-Buchanan, S. S., Clarke, D. & Mathew, A. J. Off the shelf cellular therapeutics: Factors to consider during cryopreservation and storage of human cells for clinical use. Cytotherapy 18, 697-711, doi:10.1016/j.jcyt.2016.03.295 (2016).
29 Dudley, M. E. & Rosenberg, S. A. Adoptive-cell-transfer therapy for the treatment of patients with cancer. Nat. Rev. Cancer 3, 666-675, doi:10.1038/nrc1167 (2003).
30 Boni, A. et al. Adoptive transfer of allogeneic tumor-specific T cells mediates effective regression of large tumors across major histocompatibility barriers. Blood 112, 4746-4754, doi:10.1182/blood-2008-07-169797 (2008).
31 Chambers, J. D. & Neumann, P. J. Listening to Provenge—what a costly cancer treatment says about future Medicare policy. N. Engl. J. Med. 364, 1687-1689, doi:10.1056/NEJMp1103057 (2011).
32 Brenner, M. K. Will T-cell therapy for cancer ever be a standard of care? Cancer Gene Ther. 19, 818-821, dol: 10.1038/cgt.2012.74 (2012).
33 Sheridan, C. Allogene and Celularity move CAR-T therapy off the shelf. Nat. Biotechnol. 36, 375-377, doi:10.1038/nbt0518-375 (2018).
34 Yao, J. et al. Human double negative T cells target lung cancer via ligand-dependent mechanisms that can be enhanced by IL-15, Journal for ImmunoTherapy of Cancer volume 7, Article number: 17 (2019), doi: https://doi.org/10.1186/s40425-019-0507-2.
35 Jacoby, E. et al. Murine allogeneic CD19 CAR T cells harbor potent antileukemic activity but have the potential to mediate lethal GVHD. Blood 127, 1361-1370, doi:10.1182/blood-2015-08-664250 (2016).
36 Provasi, E. et al. Editing T cell specificity towards leukemia by zinc finger nucleases and lentiviral gene transfer. Nat. Med. 18, 807-815, doi:10.1038/nm.2700 (2012).
37 Young, K. J., Kay, L. S., Phillips, M. J. & Zhang, L. Antitumor activity mediated by double-negative T cells. Cancer Res. 63, 8014-8021 (2003).
38 Young, K. J., DuTemple, B., Zhang, Z. X., Levy, G. & Zhang, L. CD4−CD8− regulatory T cells implicated in preventing graft-versus-host and promoting graft-versus-leukemia responses. Transplant. Proc. 33, 1762-1763, doi:10.1016/s0041-1345 (00) 02670-1 (2001).
39 Bian, Z. et al. Association of Epstein-Barr virus reactivation with the recovery of CD4/CD8 double-negative T lymphocytes after haploidentical hematopoietic stem cell transplantation. Bone Marrow Transplant. 52, 264-269, doi:10.1038/bmt.2016.238 (2017).
40 Suck, G. et al. NK-92: an ‘off-the-shelf therapeutic’ for adoptive natural killer cell-based cancer immunotherapy. Cancer Immunol. Immunother. 65, 485-492, doi:10.1007/s00262-015-1761-x (2016).
41 Tonn, T. et al. Treatment of patients with advanced cancer with the natural killer cell line NK-92. Cytotherapy 15, 1563-1570, doi:10.1016/j.jcyt.2013.06.017 (2013).
42 Romee, R. et al. Cytokine-induced memory-like natural killer cells exhibit enhanced responses against myeloid leukemia. Sci Transl Med 8, 357ra123, doi:10.1126/scitransimed.aaf2341 (2016).

1. A method for cryopreserving double negative T cells (DNTs), the method comprising:
a. providing a sample population of DNTs, wherein the sample population of DNTs comprises DNTs from one or more donors;
b. culturing the sample population of DNTs in a culture comprising animal serum-free media supplemented with human plasma to produce an expanded population of DNTs;
c. re-suspending the population of DNTs in a cryopreservation medium;
d. adding DMSO to the cryopreservation medium to a final concentration of between about 3% and about 15% DMSO; and
e. cryopreserving the population of DNTs in the cryopreservation medium at a temperature less than −70° C.

2. The method of claim 1, comprising adding DMSO to the cryopreservation medium to a final concentration of between about 5% and 10% DMSO.

3. The method of claim 1, wherein the sample population of DNTs comprises DNTs from two or more donors, and DNTs in the expanded population of DNTs are not alloreactive against one another.

4. The method of claim 1, wherein the culture media comprises AIM-V, GT-T551, Stemline T cell Expansion Medium, Immunocult-XF T cell Expansion Medium, Human StemXVivo, Serum-Free Human T cell Base Media, CTS T-cell Expansion SFM, Prime-XV T cell expansion XSFM, or an equivalent human T cell culture media without animal-derived components.

5. The method of claim 1, wherein the human plasma is allogeneic to the sample population of DNTs.

6. The method of claim 1, wherein the culture media comprises between about 50 and 250 IU/ml IL-2 and/or between about 0.05 and 1 μg/ml anti-CD3.

7. The method of claim 1 comprising splitting the cells in b) to maintain a cell population above 0.1 million per ml of the culture media and below 4 million per ml of the culture media and/or wherein the method comprises culturing the DNTs in b) for at least 5 days, at least 8 days, at least 10 days, at least 12 days, at least 14 days, at least 17 days, at least 20 days, or at least 25 days, optionally between 10 days and 20 days.

8. The method of claim 1, wherein the sample population of DNTs comprises DNTs from peripheral blood, leukopheresis, Leukopak, bone marrow and/or cord blood samples.

9. The method of claim 1, wherein the DNTs are at a final concentration in the cryopreservation medium of between about 2.5×10⁷ and about 2.5×10⁸ cells/ml. optionally between about 5-10×10⁷ cells/ml.

10. The method of claim 1, wherein the population of DNTs is resuspended in cryopreservation medium cooled to less than 10° C. but not frozen, optionally wherein the cryopreservation medium is cooled to about 8° C., 6° C., 4° C., or 2° C.

11. The method of claim 1, wherein the final concentration of DMSO is from about 5% to about 8.5%, optionally about 7.5%.

12. A population of DNTs cryopreserved according to the method of claim 1, wherein the DNTs express one or more surface markers, cytokines and/or chemokines.

13. The population of DNTs of claim 13, wherein the DNTs comprise surface markers (a) CD11a+, CD18+, CD10−, and/or TCR Vα24−Jα18− and/or (b) CD49d+, CD45+,CD58+, CD147+, CD98+, CD43+, CD66b−, CD35−, CD36−, and/or CD103−.

14. A method of treating cancer in a subject in need thereof, the method comprising administering to the subject the population of DNTs according to claim 13.

15. The method of claim 14, wherein the population of DNTs is expanded from one or more donors and is for administration to multiple subjects for the treatment of cancer.

16. The method of claim 14, further comprising administration of a population of allogeneic Hematopoietic Stem Cells (allo-HSCs) or peripheral blood mononuclear cells (PBMCs) to the subject, wherein the population of DNTs and HSCs or PBMCs are from the same or different donors.

17. The method of claim 14, further comprising administration of an antibody to CD3.

18. The method of claim 1, wherein the DNTs in c) are at a final concentration in the cryopreservation medium of between about 5-10×10⁷ cells/ml.

19. The method of claim 1, wherein the culture media does not comprise exogenous IL-4.
